(19) United States
(12) Patent Application Publication
(10) Pub. No.: US 2023/0242491 A1
(43) Pub. Date: Aug. 3, 2023

(54) DIMERS FROM BIOREACHABLE MOLECULES AS COPOLYMERS

(71) Applicant: Zymergen Inc., Emeryville, CA (US)

(72) Inventors: Joachim C. Ritter, San Rafael, CA (US); Arjan Zoombelt, Emeryville, CA (US); Shauna Flynn, San Francisco, CA (US); Steven M. Edgar, Albany, CA (US); John J. McNamara, El Sobrante, CA (US)

(73) Assignee: Zymergen Inc., Emeryville, CA (US)

(21) Appl. No.: 18/003,482

(22) PCT Filed: Jun. 28, 2021
(86) PCT No.: PCT/US2021/039468

§ 371 Date: Dec. 27, 2022

Related U.S. Application Data
(60) Provisional application No. 62/705,478, filed on Jun. 29, 2020.

(51) Int. Cl. C07D 241/08; C08G 59/26; C08G 59/02; C09D 163/00; C09J 7/30; C07D 487/14; C07D 405/14

(52) U.S. Cl. CPC C07D 241/08; C08G 59/26; C08G 59/027; C09D 163/00; C09J 7/30; C07D 487/14; C07D 405/14; C09J 2463/00

(57) ABSTRACT

The present disclosure relates to compositions derived from bioreachable molecules, such as amino acids or hydroxy acids. In particular, the composition can be a monomer, a polymer, or a copolymer derived from an amino acid dimer or a hydroxy acid dimer.

### CROSS-REFERENCE TO RELATED APPLICATION

This application claims the benefit of U.S. Provisional Patent Application No. 62/705,478, filed Jun. 29, 2020, which is incorporated herein by reference in its entirety.

### FIELD OF THE DISCLOSURE

The present disclosure relates to compositions derived from bioreachable molecules, such as amino acids or hydroxy acids. In particular embodiments, the composition can be a monomer, a polymer, or a copolymer derived from an amino acid dimer or a hydroxy acid dimer.

### BACKGROUND

Polymeric resins are generally high production chemicals with varying degrees of toxicity and environmental effects.

### SUMMARY

The present disclosure relates to compositions derived from bioreachable molecules having origin from biological resources. Illustrative bioreachable molecules include amino acids and hydroxy acids, which can be produced by microbes through fermentation and/or prenylation. Such bioreachable molecules can be structurally modified to provide, e.g., cyclic dimers, which in turn can be further chemically functionalized with other moieties to provide cyclic derivatives. In addition, the resulting cyclic derivatives can be employed as monomers to provide polymers or copolymers.

Accordingly, in a first aspect, the present invention encompasses a composition including a structure having formula (I) or (II):

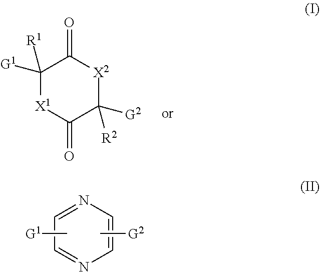

or a salt thereof, wherein each of G¹ and G² includes a reactive moiety (e.g., any described herein). In some embodiments, each of G¹ and G² includes or is, independently, hydroxyl, optionally substituted hydroxyalkyl, optionally substituted hydroxyaryl, carboxyl, optionally substituted carboxyalkyl, optionally substituted carboxyaryl, amino, optionally substituted aminoalkyl, optionally substituted aminoaryl, amido, optionally substituted amidoalkyl, cyanato, isocyanato, cyano, isocyano, optionally substituted alkenyl, optionally substituted alkynyl, optionally substituted (hetero)cycloalkyl, or optionally substituted epoxy. In other embodiments, each of R¹ and R² is, independently, H or optionally substituted alkyl. In yet other embodiments, X¹ is oxy or —N—R^{g1}, wherein R^{g1} is H, optionally substituted alkyl, optionally substituted aryl, or optionally substituted aralkyl; X² is oxy or —N—R^{g2}, wherein R^{g2} is H, optionally substituted alkyl, optionally substituted aryl, or optionally substituted aralkyl, in which R^{g1} and G¹, taken together with the nitrogen to which R^{g1} is bound, can optionally form an optionally substituted heterocyclyl; and in which R^{g2} and G², taken together with the nitrogen to which R^{g2} is bound, can optionally form an optionally substituted heterocyclyl.

In some embodiments, G¹ is -L^{G1}-L^{G3}-R^{G1}, -L^{G1}-Ar^{G1}—R^{G1}, -L^{G1}-Het^{G1}-R^{G1}, or -L^{G1}-Ar^{G1}-L^{G3}-R^{G1}; and G² is -L^{G2}-L^{G4}-R^{G2}, -L^{G2}-Ar^{G2}—R^{G2}; -L^{G2}-Het^{G2}-R^{G2}, or -L^{G2}-Ar^{G2}-L^{G4}-R^{G2} (e.g., in which L^{G1}, L^{G2}, L^{G3}, L^{G4}, Ar^{G1}; Ar^{G2}; Het^{G1}, and Het^{G2} can be any linker described herein; and in which R^{G1} and RG² can be any reactive moiety described herein).

In some embodiments, the composition includes a structure having formula (Ia), (Ib), or (Ic):

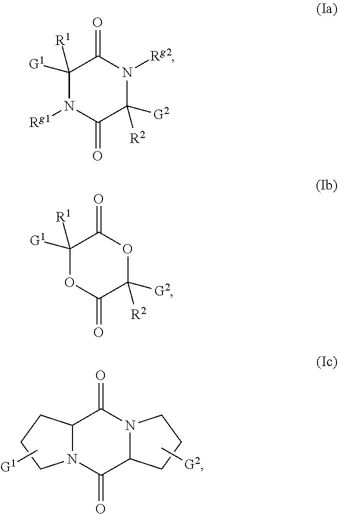

or a salt thereof, wherein each of R¹, R², R^{g1}, R^{g2}, G¹, and G² can be any described herein. In some embodiments, R^{g1} and G¹, taken together with the nitrogen to which R^{g1} is bound, and/or R^{g2} and G², taken together with the nitrogen to which R^{g2} is bound, can optionally form an optionally substituted heterocyclyl.

In some embodiments, the composition includes a structure having formula (Id):

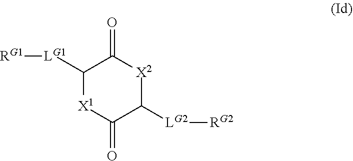

or a salt thereof, wherein each of L^{G1}, L^{G2}, X¹, X², R^{G1}, and R^{G2} can be any described herein. In yet other embodiments, the composition includes a structure having formula (Ie), (If), (Ig), (Ih), (Ii), (Ij), or a salt thereof (e.g., as described herein).

In some embodiments, the composition includes a structure selected from:

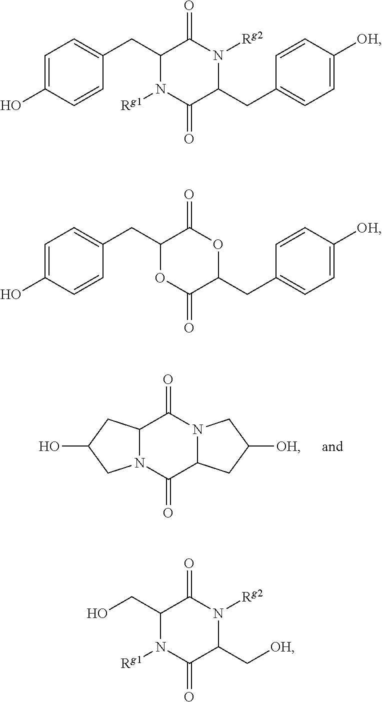

or a salt thereof, in which R^{g1} and R^{g2} can be any described herein.

In other embodiments, the composition includes a structure selected from any compound described herein (e.g., compound nos. I-1 to I-30 in Table 1), as well as a salt of any of these.

In a second aspect, the present disclosure features a method of making a composition described herein (e.g., a composition having a structure of formula (I), (Ia), (Ib), (Ic), (Id), (Ie), (If), (Ig), (Ih), (Ii), (Ij), (II), or a salt thereof). In some embodiments, the method includes providing a first amino acid and a second amino acid and forming a dimer between the first and second amino acids (e.g., thereby producing a monomer for a copolymer). In other embodiments, the first and second amino acids are selected from any amino acids and any hydroxy acids described herein. In yet other embodiments, the first and second amino acids are selected from hydroxymandelic acid, hydroxyproline, serine, and tyrosine.

In a third aspect, the present disclosure features another method of making a composition described herein (e.g., a composition having a structure of formula (I), (Ia), (Ib), (Ic), (Id), (Ie), (If), (Ig), (Ih), (Ii), (Ij), (II), or a salt thereof). In some embodiments, the method includes providing a first amino acid and a second amino acid; forming a dimer between the first and second amino acids thereby producing an ion-free epoxy resin; and optionally epoxidizing the dimer in the presence of an oxidant (e.g., any oxidant described herein). In other embodiments, the first and second amino acids are selected from any amino acids and any hydroxy acids described herein. In particular embodiments, the first and second amino acids are selected from tyrosine, tryptophan, phenylalanine, vinylglycine, allylglycine, and a derivative thereof including an optionally substituted alkenyl. In some embodiments, the first and second amino acids are selected from L-vinylglycine, L-allylglycine, O-allyl-L-tyrosine, O-buten-3-enyl-L-tryrosine, O-(3-methyl-but-2-enyl)-L-tryrosine, O-(4-methyl-pent-3-enyl)-L-tryrosine, 4-allyl-L-phenylalanine, 4-but-3-enyl-L-phenylalanine, 6-allyl-L-tryptophan, and 6-(3-methylbut-2-enyl)-L-tryptophan, or a salt thereof.

In some embodiments, the method thereby produces an ion-free epoxy resin. In other embodiments, the total ion content of the epoxy resin is less than 1 part per thousand.

In a fourth aspect, the present disclosure features yet another method of making a composition described herein (e.g., a composition having a structure of formula (I), (Ia), (Ib), (Ic), (Id), (Ie), (If), (Ig), (Ih), (Ii), (Ij), (II), or a salt thereof). In some embodiments, the method includes providing an organism a plurality of amino acids, thereby producing a plurality of prenylated amino acids; and forming a dimer between two of the plurality of amino acids.

In a fifth aspect, the present disclosure encompasses a genetically modified organism (e.g., any described herein) configured to produce any composition described herein (e.g., a composition including a structure of formula (I), (Ia), (Ib), (Ic), (Id), (Ie), (If), (Ig), (Ih), (Ii), (Ij), (II), or a salt thereof).

In a sixth aspect, the present disclosure encompasses a film (e.g., any described herein) including any composition described herein (e.g., a composition including a structure of formula (I), (Ia), (Ib), (Ic), (Id), (Ie), (If), (Ig), (Ih), (Ii), (Ij), (II), or a salt thereof). In some embodiments, the film is an adhesive or a coating.

In a seventh aspect, the present disclosure encompasses a composite or bulk structure (e.g., any described herein) including any composition described herein (e.g., a composition including a structure of formula (I), (Ia), (Ib), (Ic), (Id), (Ie), (If), (Ig), (Ih), (Ii), (Ij), (II), or a salt thereof).

In an eighth aspect, the present disclosure encompasses a fiber or a particle (e.g., any described herein) including any composition described herein (e.g., a composition including a structure of formula (I), (Ia), (Ib), (Ic), (Id), (Ie), (If), (Ig), (Ih), (Ii), (Ij), (II), or a salt thereof).

In any embodiment herein, G¹ is -L^{G1}-L^{G3}-R^{G1}, -L^{G1}-Ar^{G1}—R^{G1}, -L^{G1}-Het^{G1}-R^{G1}, or -L^{G1}-Ar^{G1}-L^{G3}-R^{G1}; and G² is -L^{G2}-L^{G4}-R^{G2}, -L^{G2}-Ar^{G2}—R^{G2}, -L^{G2}-Het^{G2}-R^{G2}, or -L^{G2}-Ar^{G2}-L^{G4}-R^{G2}, in which each of L^{G1}, L^{G2}, L^{G3}, L^{G4}, Ar^{G1}, Ar^{G2}; Het^{G1}; and Het^{G2} is a linker (e.g., any herein) and each of R^{G1} and R^{G2} is a reactive moiety (e.g., any herein). In some embodiments, each of L^{G1}, L^{G2}, L^{G3}, and L^{G4} is, independently, a covalent bond, an amide bond, —NR^{N1}— (in which R^{N1} is H or optionally substituted alkyl), a carbamate bond (e.g., a —O—C(O)—NR^{N1}— bond, in which R^{N1} is H or optionally substituted alkyl), an ester bond, oxy, carbonyl, optionally substituted alkylene, optionally substituted alkenylene, optionally substituted heteroalkylene, optionally substituted arylene, optionally substituted (aryl)(alkyl)ene, optionally substituted heterocyclyldiyl, or optionally substituted (heterocyclyl)(alkyl)ene. In other embodiments, each of Arm and Ar^{G2} includes an aryl moiety in multivalent form (e.g., as in optionally substituted arylene or optionally substituted (aryl)(alkyl)ene); and each of Hem and Het^{G2} includes a heterocyclyl moiety in multivalent form (e.g., as in optionally substituted heterocyclyldiyl or optionally substituted (heterocyclyl)(alkyl)ene). In non-limiting embodiments, each of L^{G3} and L^{G4} is, independently, a covalent bond, an amide bond, —NR^{N1}—, a carbamate bond, an ester bond, carbonyl, or oxy, wherein R^{N1} is H or optionally substituted alkyl. In yet other embodiments, each of R^{G1} and R^{G2} is, independently, hydroxyl, optionally substituted hydroxyalkyl, optionally substituted hydroxyaryl, carboxyl, optionally substituted carboxyalkyl, optionally substituted carboxyaryl, amino, optionally substituted aminoalkyl, optionally substituted aminoaryl, amido, optionally substituted amidoalkyl, cyanato, isocyanato, cyano, isocyano, optionally substituted alkenyl, optionally substituted alkynyl, optionally substituted (hetero)cycloalkyl, or optionally substituted epoxy.

In any embodiment herein, any linker herein (e.g., each of L^{G1}, L^{G2}, L^{G3}, L^{G4}, Ar^{G1}, Ar^{G2}, Het^{G1}, and Het^{G2}) is, independently, a covalent bond, an amide bond, —NR^{N1}— (in which R^{N1} is H or optionally substituted alkyl), an ester bond, oxy, carbonyl, optionally substituted alkylene, optionally substituted alkenylene, optionally substituted heteroalkylene, optionally substituted arylene, optionally substituted (aryl)(alkyl)ene, optionally substituted heterocyclyldiyl, or optionally substituted (heterocyclyl)(alkyl)ene.

In any embodiment herein, any reactive moiety (e.g., each of R^{G}, R^{G1}, and R^{G2}) is, independently, hydroxyl, optionally substituted hydroxyalkyl, optionally substituted hydroxyaryl, carboxyl, optionally substituted carboxyalkyl, optionally substituted carboxyaryl, amino, optionally substituted aminoalkyl, optionally substituted aminoaryl, amido, optionally substituted amidoalkyl, cyanato, isocyanato, cyano, isocyano, optionally substituted alkenyl, optionally substituted alkynyl, optionally substituted (hetero)cycloalkyl, or optionally substituted epoxy.

In any embodiment herein, the optionally substituted alkenyl has a structure of:

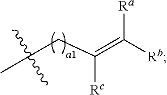

wherein each of R^{a}, R^{b}, and R^{c} is, independently, H, optionally substituted alkyl, or optionally substituted alkenyl; and wherein a1 is an integer of from 0 to 4.

In any embodiment herein, the optionally substituted epoxy has a structure of:

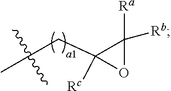

wherein each of R^{a}, R^{b}, and R^{c} is, independently, H, optionally substituted alkyl, or optionally substituted alkenyl; and wherein a1 is an integer of from 0 to 4.

In any embodiment herein, the composition includes a structure of any formula herein (e.g., formula (I), (Ia), (Ib), (Ic), (Id), (Ie), (If), (Ig), (Ih), (Ii), (Ij), and (II)) to provide a monomer, a polymer, or a copolymer. Additional details follow.

### Definitions

By “alkenyl” is meant an optionally substituted C₂₋₂₄ alkyl group having one or more double bonds. The alkenyl group can be cyclic (e.g., C₃₋₂₄ cycloalkenyl) or acyclic. The alkenyl group can also be substituted or unsubstituted. For example, the alkenyl group can be substituted with one or more substitution groups, as described herein for alkyl. Exemplary alkenyl groups include, e.g., vinyl (—CH═CH₂), vinylidene (e.g., ═C═CH₂), ethylidene (e.g., ═CH—CH₃), allyl (—CH₂—CH═CH₂), 1-propenyl (—CH═CH—CH₃), methylallyl (—CH₂—C(CH₃)═CH₂), allylidene (e.g., ═CH—CH═CH₂), homoallyl (e.g., —CH₂—CH₂—CH═CH₂), 1-butenyl (—CH═CH—CH₂—CH₃), 2-butenyl (—CH₂—CH═CH—CH₃), 3-methyl-2-butenyl or prenyl (—CH₂—CH═C(CH₃)₂), 3-butenyl (—CH₂—CH₂—CH═CH₂), 4-methyl-3-pentenyl (—CH₂—CH₂—CH═C(CH₃)₂), and the like.

By “alkenylene” is meant a multivalent (e.g., bivalent) form of an alkenyl group, as defined herein. The alkenylene group can be substituted or unsubstituted. For example, the alkenylene group can be substituted with one or more substitution groups, as described herein for alkyl. Exemplary alkenylene groups include, e.g., vinylene (—CH═CH—), vinylidene (e.g., >C═CH₂), ethanediylidene (e.g., ═CH—CH═), ethylidene (e.g., >CH—CH₃), allylidene (e.g., >CH—CH═CH₂), propenylene (e.g., —CH₂—CH═CH— or —CH₂═C═CH—), 1-propanyl-3-ylidene (═CH—CH₂—CH₂—), 2-butenylene (—CH₂—CH═CH—CH₂—), and the like.

By “alkoxy” is meant an —O-Ak group, in which Ak is an alkyl group, as defined herein.

By “alkoxyalkyl” is meant an alkyl group, as defined herein, substituted by an alkoxy group, as defined herein.

By “alkyl” and the prefix “alk” is meant a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic (e.g., C₃₋₂₄ cycloalkyl) or acyclic. The alkyl group can be branched or unbranched. The alkyl group can also be substituted or unsubstituted. For example, the alkyl group can be substituted with one, two, three or, in the case of alkyl groups of two carbons or more, four substituents independently selected from the group consisting of: (1) C₁₋₆ alkoxy (e.g., —O-Ak, wherein Ak is optionally substituted C₁₋₆ alkyl); (2) C₁₋₆ alkylsulfinyl (e.g., —S(O)-Ak, wherein Ak is optionally substituted C₁₋₆ alkyl); (3) C₁₋₆ alkylsulfonyl (e.g., —SO₂-Ak, wherein Ak is optionally substituted C₁₋₆ alkyl); (4) amino (e.g., —NR^{N1}R^{N2}, where each of R^{N1} and R^{N2} is, independently, H or optionally substituted alkyl, or R^{N1} and R^{N2}, taken together with the nitrogen atom to which each are attached, form a heterocyclyl group); (5) aryl; (6) arylalkoxy (e.g., —O-L-Ar, wherein L is a bivalent form of optionally substituted alkyl and Ar is optionally substituted aryl); (7) aryloyl (e.g., —C(O)—Ar, wherein Ar is optionally substituted aryl); (8) azido (e.g., —N₃); (9) cyano (e.g., —CN); (10) carboxyaldehyde (e.g., —C(O)H); (11) C₃₋₈ cycloalkyl (e.g., a monovalent saturated or unsaturated non-aromatic cyclic C₃₋₈ hydrocarbon group); (12) halo (e.g., F, Cl, Br, or I); (13) heterocyclyl (e.g., a 5-, 6- or 7-membered ring, unless otherwise specified, containing one, two, three, or four non-carbon heteroatoms, such as nitrogen, oxygen, phosphorous, sulfur, or halo); (14) heterocyclyloxy (e.g., —O-Het, wherein Het is heterocyclyl, as described herein); (15) heterocyclyloyl (e.g., —C(O)-Het, wherein Het is heterocyclyl, as described herein); (16) hydroxyl (e.g., —OH); (17) N-protected amino; (18) nitro (e.g., —NO₂); (19) oxo (e.g., ═O); (20) C₃₋₈ spirocyclyl (e.g., an alkylene or heteroalkylene diradical, both ends of which are bonded to the same carbon atom of the parent group); (21) C₁₋₆ thioalkoxy (e.g., —S-Ak, wherein Ak is optionally substituted C₁₋₆ alkyl); (22) thiol (e.g., —SH); (23) —CO₂R^{A}, where RA is selected from the group consisting of (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl group and Ar is optionally substituted aryl); (24) —C(O)NR^{B}R^{C}, where each of R^{B} and R^{C} is, independently, selected from the group consisting of (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl group and Ar is optionally substituted aryl); (25) —SO₂R^{D}, where R^{D} is selected from the group consisting of (a) C₁₋₆ alkyl, (b) C₄₋₁₈ aryl, and (c) (C₄₋₁₈ aryl) C₁₋₆ alkyl (e.g., -L-Ar, wherein L is a bivalent form of optionally substituted alkyl group and Ar is optionally substituted aryl); (26) —SO₂NR^{E}R^{F}, where each of R^{E} and R^{F} is, independently, selected from the group consisting of (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl group and Ar is optionally substituted aryl); and (27) —NR^{G}R^{H}, where each of R^{G} and RH is, independently, selected from the group consisting of (a) hydrogen, (b) an N-protecting group, (c) C₁₋₆ alkyl, (d) C₂₋₆ alkenyl (e.g., optionally substituted alkyl having one or more double bonds), (e) C₂₋₆ alkynyl (e.g., optionally substituted alkyl having one or more triple bonds), (f) C₄₋₁₈ aryl, (g) (C₄₋₁₈ aryl) C₁₋₆ alkyl (e.g., L-Ar, wherein L is a bivalent form of optionally substituted alkyl group and Ar is optionally substituted aryl), (h) C₃₋₈ cycloalkyl, and (i) (C₃₋₈ cycloalkyl) C₁₋₆ alkyl (e.g., -L-Cy, wherein L is a bivalent form of optionally substituted alkyl group and Cy is optionally substituted cycloalkyl, as described herein), wherein in one embodiment no two groups are bound to the nitrogen atom through a carbonyl group or a sulfonyl group. The alkyl group can be a primary, secondary, or tertiary alkyl group substituted with one or more substituents (e.g., one or more halo or alkoxy). In some embodiments, the unsubstituted alkyl group is a C₁₋₃, C₁₋₆, C₁₋₁₂, C₁₋₁₆, C₁₋₁₈, C₁₋₂₀, C₁₋₂₄, C₂₋₃, C₂₋₆, C₂₋₁₂, C₂₋₁₆, C₂₋₁₈, C₂₋₂₀, C₂₋₂₄, C₃₋₆, C₃₋₁₂, C₃₋₁₆, C₃₋₁₈, C₃₋₂₀, C₃₋₂₄, C₄₋₆, C₄₋₁₂, C₄₋₁₆, C₄₋₁₈, C₄₋₂₀, C₄₋₂₄, C₅₋₆, C₅₋₁₂, C₅₋₁₆, C₅₋₁₈, C₅₋₂₀, C₅₋₂₄, C₆₋₁₂, C₆₋₁₆, C₆₋₁₈, C₆₋₂₀, C₆₋₂₄, C₇₋₁₂, C₇₋₁₆, C₇₋₁₈, C₇₋₂₀, C₇₋₂₄, C₈₋₁₂, C₈₋₁₆, C₈₋₁₈, C₈₋₂₀, C₈₋₂₄, C₉₋₁₂, C₉₋₁₆, C₉₋₁₈, C₉₋₂₀, C₉₋₂₄, C₁₀₋₁₂, C₁₀₋₁₆, C₁₀₋₁₈, C₁₀₋₂₀, or C₁₋₂₄ alkyl group.

By “alkylene” is meant a multivalent (e.g., bivalent) form of an alkyl group, as described herein. Exemplary alkylene groups include methylene, ethylene, propylene, butylene, etc. In some embodiments, the alkylene group is a C₁₋₃, C₁₋₆, C₁₋₁₂, C₁₋₁₆, C₁₋₁₈, C₁₋₂₀, C₁₋₂₄, C₂₋₃, C₂₋₆, C₂₋₁₂, C₂₋₁₆, C₂₋₁₈, C₂₋₂₀, or C₂₋₂₄ alkylene group. The alkylene group can be branched or unbranched. The alkylene group can also be substituted or unsubstituted. For example, the alkylene group can be substituted with one or more substitution groups, as described herein for alkyl. Exemplary alkylene groups include, e.g., methylene (e.g., ═CH₂, >CH₂, or —CH₂—), ethylene (—CH₂—CH₂—), propylene (e.g., —CH(CH₃)—CH₂— or —CH₂—CH₂—CH₂—), and the like.

By “alkynyl” is meant an optionally substituted C₂₋₂₄ alkyl group having one or more triple bonds. The alkynyl group can be cyclic or acyclic and is exemplified by ethynyl, 1-propynyl, and the like. The alkynyl group can also be substituted or unsubstituted. For example, the alkynyl group can be substituted with one or more substitution groups, as described herein for alkyl.

By “amide bond” is meant —C(O)NR^{N1}— or —NR^{N1}C(O)—, where R^{N1} is H or optionally substituted alkyl. A non-limiting amide bond includes —C(O)NH—.

By “amido” is meant —C(O)NR^{N1}R^{N2}, where each of R^{N1} and R^{N2} is, independently, H or optionally substituted alkyl, or R^{N1} and R^{N2}, taken together with the nitrogen atom to which each are attached, form a heterocyclyl group, as defined herein.

By “amidoalkyl” is meant an alkyl group, as defined herein, substituted by an amido group, as defined herein.

By “amino” is meant —NR^{N1}R^{N2}, where each of R^{N1} and R^{N2} is, independently, H, optionally substituted alkyl, or optionally substituted aryl, or R^{N1} and R^{N2}, taken together with the nitrogen atom to which each are attached, form a heterocyclyl group, as defined herein.

By “aminoalkyl” is meant an alkyl group, as defined herein, substituted by an amino group, as defined herein.

By “aminoaryl” is meant an aryl group, as defined herein, substituted by an amino group, as defined herein.

By “aralkyl” or “arylalkyl” is meant -Ak-Ar, in which Ak is an optionally substituted alkylene, as defined herein, and Ar is an optionally substituted aryl, as defined herein. The aralkyl group can be substituted or unsubstituted. For example, the aralkyl group can be substituted with one or more substitution groups, as described herein for aryl and/or alkyl. Non-limiting unsubstituted aralkyl groups are of from 7 to 16 carbons (C₇₋₁₆ aralkyl), as well as those having an aryl group with 4 to 18 carbons and an alkylene group with 1 to 6 carbons (i.e., —C₁₋₆ alkylene-C₄₋₁₈ aryl).

By “aryl” is meant a group that contains any carbon-based aromatic group including, but not limited to, phenyl, benzyl, anthracenyl, anthryl, benzocyclobutenyl, benzocyclooctenyl, biphenylyl, chrysenyl, dihydroindenyl, fluoranthenyl, indacenyl, indenyl, naphthyl, phenanthryl, phenoxybenzyl, picenyl, pyrenyl, terphenyl, and the like, including fused benzo-C₄₋₈ cycloalkyl radicals (e.g., as defined herein) such as, for instance, indanyl, tetrahydronaphthyl, fluorenyl, and the like. The term aryl also includes heteroaryl, which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term non-heteroaryl, which is also included in the term aryl, defines a group that contains an aromatic group that does not contain a heteroatom. The aryl group can be substituted or unsubstituted. The aryl group can be substituted with one, two, three, four, or five substituents independently selected from the group consisting of: (1) C₁₋₆ alkanoyl (e.g., —C(O)-Ak, wherein Ak is optionally substituted C₁₋₆ alkyl); (2) C₁₋₆ alkyl; (3) C₁₋₆ alkoxy (e.g., —O-Ak, wherein Ak is optionally substituted C₁₋₆ alkyl); (4) C₁₋₆ alkoxy-C₁₋₆ alkyl (e.g., -L-O-Ak, wherein L is a bivalent form of optionally substituted alkyl group and Ak is optionally substituted C₁₋₆ alkyl); (5) C₁₋₆ alkylsulfinyl (e.g., —S(O)-Ak, wherein Ak is optionally substituted C₁₋₆ alkyl); (6) C₁₋₆ alkylsulfinyl-C₁₋₆ alkyl (e.g., -L-S(O)-Ak, wherein L is a bivalent form of optionally substituted alkyl group and Ak is optionally substituted C₁₋₆ alkyl); (7) C₁₋₆ alkylsulfonyl (e.g., —SO₂-Ak, wherein Ak is optionally substituted C₁₋₆ alkyl); (8) C₁₋₆ alkylsulfonyl-C₁₋₆ alkyl (e.g., -L-SO₂-Ak, wherein L is a bivalent form of optionally substituted alkyl group and Ak is optionally substituted C₁₋₆ alkyl); (9) aryl; (10) amino (e.g., —NR^{N1}R^{N2}, where each of R^{N1} and R^{N2} is, independently, H or optionally substituted alkyl, or R^{N1} and R^{N2}, taken together with the nitrogen atom to which each are attached, form a heterocyclyl group); (11) C₁₋₆ aminoalkyl (e.g., an alkyl group, as defined herein, substituted by one or more —NR^{N1}R^{N2} groups, as described herein); (12) heteroaryl (e.g., a subset of heterocyclyl groups (e.g., a 5-, 6- or 7-membered ring, unless otherwise specified, containing one, two, three, or four non-carbon heteroatoms), which are aromatic); (13) (C₄₋₁₈ aryl) C₁₋₆ alkyl (e.g., -L-Ar, wherein L is a bivalent form of optionally substituted alkyl and Ar is optionally substituted aryl); (14) aryloyl (e.g., —C(O)—Ar, wherein Ar is optionally substituted aryl); (15) azido (e.g., —N₃); (16) cyano (e.g., —CN); (17) C₁₋₆ azidoalkyl (e.g., an alkyl group, as defined herein, substituted by one or more azido groups, as described herein); (18) carboxyaldehyde (e.g., —C(O)H); (19) carboxyaldehyde-C₁₋₆ alkyl (e.g., an alkyl group, as defined herein, substituted by one or more carboxyaldehyde groups, as described herein); (20) C₃₋₈ cycloalkyl (e.g., a monovalent saturated or unsaturated non-aromatic cyclic C₃₋₈ hydrocarbon group); (21) (C₃₋₈ cycloalkyl) C₁₋₆ alkyl (e.g., an alkyl group, as defined herein, substituted by one or more cycloalkyl groups, as described herein); (22) halo (e.g., F, Cl, Br, or I); (23) C₁₋₆ haloalkyl (e.g., an alkyl group, as defined herein, substituted by one or more halo groups, as described herein); (24) heterocyclyl (e.g., a 5-, 6- or 7-membered ring, unless otherwise specified, containing one, two, three, or four non-carbon heteroatoms, such as nitrogen, oxygen, phosphorous, sulfur, or halo); (25) heterocyclyloxy (e.g., —O-Het, wherein Het is heterocyclyl, as described herein); (26) heterocyclyloyl (e.g., —C(O)-Het, wherein Het is heterocyclyl, as described herein); (27) hydroxyl (e.g., —OH); (28) C₁₋₆ hydroxyalkyl (e.g., an alkyl group, as defined herein, substituted by one or more hydroxyl, as described herein); (29) nitro (e.g., —NO₂); (30) C₁₋₆ nitroalkyl (e.g., an alkyl group, as defined herein, substituted by one or more nitro, as described herein); (31) N-protected amino; (32) N-protected amino-C₁₋₆ alkyl (e.g., an alkyl group, as defined herein, substituted by one or more N-protected amino groups); (33) oxo (e.g., ═O); (34) C₁₋₆ thioalkoxy (e.g., —S-Ak, wherein Ak is optionally substituted C₁₋₆ alkyl); (35) thio-C₁₋₆ alkoxy-C₁₋₆ alkyl (e.g., -L-S-Ak, wherein L is a bivalent form of optionally substituted alkyl and Ak is optionally substituted C₁₋₆ alkyl); (36) —(CH₂)ᵣCO₂R^{A}, where r is an integer of from zero to four, and R^{A} is selected from the group consisting of (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl and Ar is optionally substituted aryl); (37) —(CH₂)ᵣCONR^{B}R^{C}, where r is an integer of from zero to four and where each R^{B} and R^{c} is independently selected from the group consisting of (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl and Ar is optionally substituted aryl); (38) —(CH₂)ᵣSO₂R^{D}, where r is an integer of from zero to four and where R^{D} is selected from the group consisting of (a) C₁₋₆ alkyl, (b) C₄₋₁₈ aryl, and (c) (C₄₋₁₈ aryl) C₁₋₆ alkyl (e.g., -L-Ar, wherein L is a bivalent form of optionally substituted alkyl and Ar is optionally substituted aryl); (39) —(CH₂)ᵣSO₂NR^{E}R^{F}, where r is an integer of from zero to four and where each of R^{E} and R^{F} is, independently, selected from the group consisting of (a) hydrogen, (b) C₁₋₆ alkyl, (c) C₄₋₁₈ aryl, and (d) (C₄₋₁₈ aryl) C₁₋₆ alkyl (e.g., -L-Ar, wherein Lisa bivalent form of optionally substituted alkyl and Ar is optionally substituted aryl); (40) —(CH₂)ᵣNR^{G}R^{H}, where r is an integer of from zero to four and where each of R^{G} and RH is, independently, selected from the group consisting of (a) hydrogen, (b) an N-protecting group, (c) C₁₋₆ alkyl, (d) C₂₋₆ alkenyl (e.g., optionally substituted alkyl having one or more double bonds), (e) C₂₋₆ alkynyl (e.g., optionally substituted alkyl having one or more triple bonds), (f) C₄₋₁₈ aryl, (g) (C₄₋₁₈ aryl) C₁₋₆ alkyl (e.g., -L-Ar, wherein L is a bivalent form of optionally substituted alkyl and Ar is optionally substituted aryl), (h) C₃₋₈ cycloalkyl, and (i) (C₃₋₈ cycloalkyl) C₁₋₆ alkyl (e.g., -L-Cy, wherein L is a bivalent form of optionally substituted alkyl and Cy is optionally substituted cycloalkyl, as described herein), wherein in one embodiment no two groups are bound to the nitrogen atom through a carbonyl group or a sulfonyl group; (41) thiol (e.g., —SH); (42) perfluoroalkyl (e.g., an alkyl group having each hydrogen atom substituted with a fluorine atom); (43) perfluoroalkoxy (e.g., —OR^{f}, where R^{f} is an alkyl group having each hydrogen atom substituted with a fluorine atom); (44) aryloxy (e.g., —OAr, where Ar is optionally substituted aryl); (45) cycloalkoxy (e.g., —O-Cy, wherein Cy is optionally substituted cycloalkyl, as described herein); (46) cycloalkylalkoxy (e.g., —O-L-Cy, wherein L is a bivalent form of optionally substituted alkyl and Cy is optionally substituted cycloalkyl, as described herein); and (47) arylalkoxy (e.g., —O-L-Ar, wherein L is a bivalent form of optionally substituted alkyl and Ar is optionally substituted aryl). In particular embodiments, an unsubstituted aryl group is a C₄₋₁₈, C₄₋₁₄, C₄₋₁₂, C₄₋₁₀, C₆₋₁₈, C₆₋₁₄, C₆₋₁₂, or C₆₋₁₀ aryl group.

By “(aryl)(alkyl)ene” is meant a bivalent form including an arylene group, as described herein, attached to an alkylene or a heteroalkylene group, as described herein. In some embodiments, the (aryl)(alkyl)ene group is -L-Ar— or -L-Ar-L- or —Ar-L-, in which Ar is an arylene group and each L is, independently, an optionally substituted alkylene group or an optionally substituted heteroalkylene group.

By “arylene” is meant a multivalent (e.g., bivalent) form of an aryl group, as described herein. Non-limiting arylene groups include phenylene, naphthylene, biphenylene, triphenylene, diphenyl ether, acenaphthenylene, anthrylene, or phenanthrylene. In some embodiments, the arylene group is a C₄₋₁₈, C₄₋₁₄, C₄₋₁₂, C₄₋₁₀, C₆₋₁₈, C₆₋₁₄, C₆₋₁₂, or C₆₋₁₀ arylene group. The arylene group can be branched or unbranched. The arylene group can also be substituted or unsubstituted. For example, the arylene group can be substituted with one or more substitution groups, as described herein for aryl.

By “carboxyl” is meant a —CO₂H group.

By “carboxyalkyl” is meant an alkyl group, as defined herein, substituted by a carboxyl group, as defined herein.

By “carboxyaryl” is meant an aryl group, as described herein, substituted with one or more —CO₂H groups.

By “cyanato” is meant a —OCN group.

By “cyano” is meant a —CN group.

By “cycloalkyl” is meant a monovalent saturated or unsaturated non-aromatic cyclic hydrocarbon group of from three to eight carbons, unless otherwise specified, and is exemplified by cyclopropyl, cyclobutyl, cyclopentyl, cyclohexyl, cycloheptyl, bicyclo[2.2.1.]heptyl, and the like. The cycloalkyl group can also be substituted or unsubstituted. For example, the cycloalkyl group can be substituted with one or more groups including those described herein for alkyl. Exemplary cycloalkyl groups include C₃₋₆ cycloalkyl and C₃₋₈ cycloalkyl.

By “epoxy” is meant -L-RH, in which L is a linker and RH is an optionally substituted oxiranyl group. In particular embodiments, L can be a bond, optionally substituted alkylene, or optionally substituted heteroalkylene; and RH can be monovalent, saturated cycloalkyl group having one oxygen atom and two carbon atoms, and in which each carbon atom can include hydrogen atoms or the hydrogen atoms can be substituted with one or more groups including those described herein for alkyl. In other embodiments, RH is an optionally substituted 2-oxiranyl, in which each H in

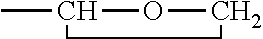

can be substituted with one or more substituents described herein for alkyl.

By “ester bond” is meant is meant —C(O)O— or —OC(O)—.

By “halo” is meant F, Cl, Br, or I.

By “haloalkyl” is meant an alkyl group, as defined herein, substituted with one or more halo.

By “heteroalkylene” is meant a bivalent form of an alkylene group, as defined herein, containing one, two, three, or four non-carbon heteroatoms (e.g., independently selected from the group consisting of nitrogen, oxygen, phosphorous, sulfur, selenium, or halo). The heteroalkylene group can be substituted or unsubstituted. For example, the heteroalkylene group can be substituted with one or more substitution groups, as described herein for alkyl. Non-limiting heteroalkylene groups include, e.g., —O-Ak-, -Ak-O—, —S-Ak-, or -Ak-S—, in which Ak is an optionally substituted alkylene, as described herein.

By “(hetero)cycloalkyl” is meant a saturated cycloalkyl group, as defined herein, having one or more carbon atoms may be replaced by a non-carbon heteroatom (e.g., N, O or S). Examples of (hetero)cycloalkyl are oxiranyl (e.g., 2-oxiranyl, such as

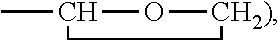

oxetanyl (e.g., 3-oxetanyl, such as

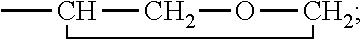

or 2-oxetanyl

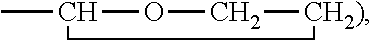

and tetrahydrofuryl (e.g., 2-tetrahydrofuryl or 3-tetrahydrofuryl). The (hetero)cycloalkyl group can also be substituted or unsubstituted. For example, the (hetero)cycloalkyl group can be substituted with one or more groups including those described herein for alkyl. Exemplary (hetero)cycloalkyl groups include C₂₋₆ cycloalkyl and C₂₋₈ cycloalkyl. Yet other exemplary (hetero)cycloalkyl groups include a cyclic ether group, in which the non-carbon heteroatom is O.

By “heterocyclyl” is meant a 3-, 4-, 5-, 6- or 7-membered ring (e.g., a 5-, 6-, or 7-membered ring), unless otherwise specified, containing one, two, three, or four non-carbon heteroatoms (e.g., independently selected from the group consisting of nitrogen, oxygen, phosphorous, sulfur, selenium, or halo). The 3-membered ring has zero to one double bonds, the 4- and 5-membered ring has zero to two double bonds, and the 6- and 7-membered rings have zero to three double bonds. The term “heterocyclyl” also includes bicyclic, tricyclic and tetracyclic groups in which any of the above heterocyclic rings is fused to one, two, or three rings independently selected from the group consisting of an aryl ring, a cyclohexane ring, a cyclohexene ring, a cyclopentane ring, a cyclopentene ring, and another monocyclic heterocyclic ring, such as indolyl, quinolyl, isoquinolyl, tetrahydroquinolyl, benzofuryl, benzothienyl and the like. Heterocyclics include acridinyl, adenyl, alloxazinyl, azaadamantanyl, azabenzimidazolyl, azabicyclononyl, azacycloheptyl, azacyclooctyl, azacyclononyl, azahypoxanthinyl, azaindazolyl, azaindolyl, azecinyl, azepanyl, azepinyl, azetidinyl, azetyl, aziridinyl, azirinyl, azocanyl, azocinyl, azonanyl, benzimidazolyl, benzisothiazolyl, benzisoxazolyl, benzodiazepinyl, benzodiazocinyl, benzodihydrofuryl, benzodioxepinyl, benzodioxinyl, benzodioxanyl, benzodioxocinyl, benzodioxolyl, benzodithiepinyl, benzodithiinyl, benzodioxocinyl, benzofuranyl, benzophenazinyl, benzopyranonyl, benzopyranyl, benzopyrenyl, benzopyronyl, benzoquinolinyl, benzoquinolizinyl, benzothiadiazepinyl, benzothiadiazolyl, benzothiazepinyl, benzothiazocinyl, benzothiazolyl, benzothienyl, benzothiophenyl, benzothiazinonyl, benzothiazinyl, benzothiopyranyl, benzothiopyronyl, benzotriazepinyl, benzotriazinonyl, benzotriazinyl, benzotriazolyl, benzoxathiinyl, benzotrioxepinyl, benzoxadiazepinyl, benzoxathiazepinyl, benzoxathiepinyl, benzoxathiocinyl, benzoxazepinyl, benzoxazinyl, benzoxazocinyl, benzoxazolinonyl, benzoxazolinyl, benzoxazolyl, benzylsultamyl benzylsultimyl, bipyrazinyl, bipyridinyl, carbazolyl (e.g., 4H-carbazolyl), carbolinyl (e.g., β-carbolinyl), chromanonyl, chromanyl, chromenyl, cinnolinyl, coumarinyl, cytdinyl, cytosinyl, decahydroisoquinolinyl, decahydroquinolinyl, diazabicyclooctyl, diazetyl, diaziridinethionyl, diaziridinonyl, diaziridinyl, diazirinyl, dibenzisoquinolinyl, dibenzoacridinyl, dibenzocarbazolyl, dibenzofuranyl, dibenzophenazinyl, dibenzopyranonyl, dibenzopyronyl (xanthonyl), dibenzoquinoxalinyl, dibenzothiazepinyl, dibenzothiepinyl, dibenzothiophenyl, dibenzoxepinyl, dihydroazepinyl, dihydroazetyl, dihydrofuranyl, dihydrofuryl, dihydroisoquinolinyl, dihydropyranyl, dihydropyridinyl, dihydroypyridyl, dihydroquinolinyl, dihydrothienyl, dihydroindolyl, dioxanyl, dioxazinyl, dioxindolyl, dioxiranyl, dioxenyl, dioxinyl, dioxobenzofuranyl, dioxolyl, dioxotetrahydrofuranyl, dioxothiomorpholinyl, dithianyl, dithiazolyl, dithienyl, dithiinyl, furanyl, furazanyl, furoyl, furyl, guaninyl, homopiperazinyl, homopiperidinyl, hypoxanthinyl, hydantoinyl, imidazolidinyl, imidazolinyl, imidazolyl, indazolyl (e.g., 1H-indazolyl), indolenyl, indolinyl, indolizinyl, indolyl (e.g., 1H-indolyl or 3H-indolyl), isatinyl, isatyl, isobenzofuranyl, isochromanyl, isochromenyl, isoindazoyl, isoindolinyl, isoindolyl, isopyrazolonyl, isopyrazolyl, isoxazolidiniyl, isoxazolyl, isoquinolinyl, isoquinolinyl, isothiazolidinyl, isothiazolyl, morpholinyl, naphthindazolyl, naphthindolyl, naphthiridinyl, naphthopyranyl, naphthothiazolyl, naphthothioxolyl, naphthotriazolyl, naphthoxindolyl, naphthyridinyl, octahydroisoquinolinyl, oxabicycloheptyl, oxauracil, oxadiazolyl, oxazinyl, oxaziridinyl, oxazolidinyl, oxazolidonyl, oxazolinyl, oxazolonyl, oxazolyl, oxepanyl, oxetanonyl, oxetanyl, oxetyl, oxtenayl, oxindolyl, oxiranyl, oxobenzoisothiazolyl, oxochromenyl, oxoisoquinolinyl, oxoquinolinyl, oxothiolanyl, phenanthridinyl, phenanthrolinyl, phenazinyl, phenothiazinyl, phenothienyl (benzothiofuranyl), phenoxathiinyl, phenoxazinyl, phthalazinyl, phthalazonyl, phthalidyl, phthalimidinyl, piperazinyl, piperidinyl, piperidonyl (e.g., 4-piperidonyl), pteridinyl, purinyl, pyranyl, pyrazinyl, pyrazolidinyl, pyrazolinyl, pyrazolopyrimidinyl, pyrazolyl, pyridazinyl, pyridinyl, pyridopyrazinyl, pyridopyrimidinyl, pyridyl, pyrimidinyl, pyrimidyl, pyronyl, pyrrolidinyl, pyrrolidonyl (e.g., 2-pyrrolidonyl), pyrrolinyl, pyrrolizidinyl, pyrrolyl (e.g., 2H-pyrrolyl), quinazolinyl, quinolinyl, quinolizinyl (e.g., 4H-quinolizinyl), quinoxalinyl, quinuclidinyl, selenazinyl, selenazolyl, selenophenyl, succinimidyl, sulfolanyl, tetrahydrofuranyl, tetrahydrofuryl, tetrahydroisoquinolinyl, tetrahydroisoquinolyl, tetrahydropyridinyl, tetrahydropyridyl (piperidyl), tetrahydropyranyl, tetrahydropyronyl, tetrahydroquinolinyl, tetrahydroquinolyl, tetrahydrothienyl, tetrahydrothiophenyl, tetrazinyl, tetrazolyl, thiadiazinyl (e.g., 6H-1,2,5-thiadiazinyl or 2H,6H-1,5,2-dithiazinyl), thiadiazolyl, thianthrenyl, thianyl, thianaphthenyl, thiazepinyl, thiazinyl, thiazolidinedionyl, thiazolidinyl, thiazolyl, thienyl, thiepanyl, thiepinyl, thietanyl, thietyl, thiiranyl, thiocanyl, thiochromanonyl, thiochromanyl, thiochromenyl, thiodiazinyl, thiodiazolyl, thioindoxyl, thiomorpholinyl, thiophenyl, thiopyranyl, thiopyronyl, thiotriazolyl, thiourazolyl, thioxanyl, thioxolyl, thymidinyl, thyminyl, triazinyl, triazolyl, trithianyl, urazinyl, urazolyl, uretidinyl, uretinyl, uricyl, uridinyl, xanthenyl, xanthinyl, xanthionyl, and the like, as well as modified forms thereof (e.g., including one or more oxo and/or amino), and salts thereof. The heterocyclyl group can be substituted or unsubstituted. For example, the heterocyclyl group can be substituted with one or more substitution groups, as described herein for aryl.

By “(heterocyclyl)(alkyl)ene” is meant a bivalent form including a heterocyclyldiyl group, as described herein, attached to an alkylene or a heteroalkylene group, as described herein. In some embodiments, the (heterocyclyl)(alkyl)ene group is -L-Het-, -L-Het-L-, or -Het-L-, in which Het is a heterocyclyldiyl group and L is an optionally substituted alkylene group or an optionally substituted heteroalkylene group.

By “heterocyclyldiyl” is meant a bivalent form of a heterocyclyl group, as described herein. In one instance, the heterocyclyldiyl is formed by removing a hydrogen from a heterocyclyl group. Exemplary heterocyclyldiyl groups include piperdylidene, quinolinediyl, etc. The heterocyclyldiyl group can also be substituted or unsubstituted. For example, the heterocyclyldiyl group can be substituted with one or more substitution groups, as described herein for heterocyclyl.

By “hydroxyl” is meant —OH.

By “hydroxyalkyl” is meant an alkyl group, as defined herein, substituted by one to three hydroxyl groups, with the proviso that no more than one hydroxyl group may be attached to a single carbon atom of the alkyl group and is exemplified by hydroxymethyl, dihydroxypropyl, and the like.

By “hydroxyaryl” is meant an aryl group, as defined herein, substituted by one to three hydroxyl groups, with the proviso that no more than one hydroxyl group may be attached to a single carbon atom of the aryl group and is exemplified by hydroxyphenyl, dihydroxyphenyl, and the like.

By “hydroxyaralkyl” is meant an aralkyl group, as defined herein, substituted by one to three hydroxyl groups, with the proviso that no more than one hydroxyl group may be attached to a single carbon atom of the aryl group and is exemplified by hydroxybenzyl, dihydroxybenzyl, and the like.

By “isocyanato” is meant a —NCO group.

By “isocyano” is meant a —NC group.

By “oxy” is meant —O—.

By “protecting group” is meant any group intended to protect a reactive group against undesirable synthetic reactions. Commonly used protecting groups are disclosed in “Greene's Protective Groups in Organic Synthesis,” John Wiley & Sons, New York, 2007 (4th ed., eds. P. G. M. Wuts and T. W. Greene), which is incorporated herein by reference. O-protecting groups include an optionally substituted alkyl group (e.g., forming an ether with reactive group O), such as methyl, methoxymethyl, methylthiomethyl, benzoyloxymethyl, t-butoxymethyl, etc.; an optionally substituted alkanoyl group (e.g., forming an ester with the reactive group O), such as formyl, acetyl, chloroacetyl, fluoroacetyl (e.g., perfluoroacetyl), methoxyacetyl, pivaloyl, t-butylacetyl, phenoxyacetyl, etc.; an optionally substituted aryloyl group (e.g., forming an ester with the reactive group O), such as —C(O)—Ar, including benzoyl; an optionally substituted alkylsulfonyl group (e.g., forming an alkylsulfonate with reactive group O), such as —SO₂—R^{S1}, where R^{S1} is optionally substituted C₁₋₁₂ alkyl, such as mesyl or benzylsulfonyl; an optionally substituted arylsulfonyl group (e.g., forming an arylsulfonate with reactive group O), such as —SO₂—R^{S4}, where R^{S4} is optionally substituted C₄₋₁₈ aryl, such as tosyl or phenylsulfonyl; an optionally substituted alkoxycarbonyl or aryloxycarbonyl group (e.g., forming a carbonate with reactive group O), such as —C(O)—OR^{T1}, where R^{T1} is optionally substituted C₁₋₁₂ alkyl or optionally substituted C₄₋₁₈ aryl, such as methoxycarbonyl, methoxymethylcarbonyl, t-butyloxycarbonyl (Boc), or benzyloxycarbonyl (Cbz); or an optionally substituted silyl group (e.g., forming a silyl ether with reactive group O), such as —Si—(R^{T2})₃, where each R^{T2} is, independently, optionally substituted C₁₋₁₂ alkyl or optionally substituted C₄₋₁₈ aryl, such as trimethylsilyl, t-butyldimethylsilyl, or t-butyldiphenylsilyl. N-protecting groups include, e.g., formyl, acetyl, benzoyl, pivaloyl, t-butylacetyl, alanyl, phenylsulfonyl, benzyl, Boc, and Cbz. Such protecting groups can employ any useful agent to cleave the protecting group, thereby restoring the reactivity of the unprotected reactive group.

By “salt” is meant an ionic form of a compound or structure (e.g., any formulas, compounds, or compositions described herein), which includes a cation or anion compound to form an electrically neutral compound or structure. Salts are well known in the art. For example, non-toxic salts are described in Berge S M et al., “Pharmaceutical salts,” J. Pharm. Sci. 1977 January; 66(1):1-19; and in “Handbook of Pharmaceutical Salts: Properties, Selection, and Use,” Wiley-VCH, April 2011 (2nd rev. ed., eds. P. H. Stahl and C. G. Wermuth. The salts can be prepared in situ during the final isolation and purification of the compounds of the invention or separately by reacting the free base group with a suitable organic acid (thereby producing an anionic salt) or by reacting the acid group with a suitable metal or organic salt (thereby producing a cationic salt). Representative anionic salts include acetate, adipate, alginate, ascorbate, aspartate, benzenesulfonate, benzoate, bicarbonate, bisulfate, bitartrate, borate, bromide, butyrate, camphorate, camphorsulfonate, chloride, citrate, cyclopentanepropionate, digluconate, dihydrochloride, diphosphate, dodecylsulfate, edetate, ethanesulfonate, fumarate, glucoheptonate, gluconate, glutamate, glycerophosphate, hemisulfate, heptonate, hexanoate, hydrobromide, hydrochloride, hydroiodide, hydroxyethanesulfonate, hydroxynaphthoate, iodide, lactate, lactobionate, laurate, lauryl sulfate, malate, maleate, malonate, mandelate, mesylate, methanesulfonate, methylbromide, methylnitrate, methylsulfate, mucate, 2-naphthalenesulfonate, nicotinate, nitrate, oleate, oxalate, palmitate, pamoate, pectinate, persulfate, 3-phenylpropionate, phosphate, picrate, pivalate, polygalacturonate, propionate, salicylate, stearate, subacetate, succinate, sulfate, tannate, tartrate, theophyllinate, thiocyanate, triethiodide, toluenesulfonate, undecanoate, valerate salts, and the like. Representative cationic salts include metal salts, such as alkali or alkaline earth salts, e.g., barium, calcium (e.g., calcium edetate), lithium, magnesium, potassium, sodium, and the like; other metal salts, such as aluminum, bismuth, iron, and zinc; as well as nontoxic ammonium, quaternary ammonium, and amine cations, including, but not limited to ammonium, tetramethylammonium, tetraethylammonium, methylamine, dimethylamine, trimethylamine, triethylamine, ethylamine, pyridinium, and the like. Other cationic salts include organic salts, such as chloroprocaine, choline, dibenzylethylenediamine, diethanolamine, ethylenediamine, methylglucamine, and procaine. Yet other salts include ammonium, sulfonium, sulfoxonium, phosphonium, iminium, imidazolium, benzimidazolium, amidinium, guanidinium, phosphazinium, phosphazenium, pyridinium, etc., as well as other cationic groups described herein (e.g., optionally substituted isoxazolium, optionally substituted oxazolium, optionally substituted thiazolium, optionally substituted pyrrolium, optionally substituted furanium, optionally substituted thiophenium, optionally substituted imidazolium, optionally substituted pyrazolium, optionally substituted isothiazolium, optionally substituted triazolium, optionally substituted tetrazolium, optionally substituted furazanium, optionally substituted pyridinium, optionally substituted pyrimidinium, optionally substituted pyrazinium, optionally substituted triazinium, optionally substituted tetrazinium, optionally substituted pyridazinium, optionally substituted oxazinium, optionally substituted pyrrolidinium, optionally substituted pyrazolidinium, optionally substituted imidazolinium, optionally substituted isoxazolidinium, optionally substituted oxazolidinium, optionally substituted piperazinium, optionally substituted piperidinium, optionally substituted morpholinium, optionally substituted azepanium, optionally substituted azepinium, optionally substituted indolium, optionally substituted isoindolium, optionally substituted indolizinium, optionally substituted indazolium, optionally substituted benzimidazolium, optionally substituted isoquinolinum, optionally substituted quinolizinium, optionally substituted dehydroquinolizinium, optionally substituted quinolinium, optionally substituted isoindolinium, optionally substituted benzimidazolinium, and optionally substituted purinium).

By “stereo isomer” is meant any of the various stereoisomeric configurations that may exist for a given compound of the present invention and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the disclosure includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereomers” are stereoisomers that have at least two asymmetric atoms, but which are not mirror-images of each other. The absolute stereochemistry can be specified according to the Cahn-Ingold-Prelog R-S system.

By “attaching,” “attachment,” or related word forms is meant any covalent or non-covalent bonding interaction between two components. Non-covalent bonding interactions include, without limitation, hydrogen bonding, ionic interactions, halogen bonding, electrostatic interactions, it bond interactions, hydrophobic interactions, inclusion complexes, clathration, van der Waals interactions, and combinations thereof.

As used herein, the term “about” means +/−10% of any recited value. As used herein, this term modifies any recited value, range of values, or endpoints of one or more ranges.

As used herein, the terms “top,” “bottom,” “upper,” “lower,” “above,” and “below” are used to provide a relative relationship between structures. The use of these terms does not indicate or require that a particular structure must be located at a particular location in the apparatus.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

This written description uses examples to disclose the embodiments, including the best mode, and also to enable those of ordinary skill in the art to make and use the invention. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Note that not all of the activities described above in the general description or the examples are required, that a portion of a specific activity may not be required, and that one or more further activities may be performed in addition to those described. The order in which activities are listed is not necessarily the order in which they are performed.

In this specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

Benefits, other advantages, and solutions to problems have been described herein with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

### BRIEF DESCRIPTION OF THE DRAWINGS

FIG. 1 shows a schematic of illustrative cyclic derivatives formed from bioreachable molecules.

FIG. 2 shows an illustrative schematic of modifying a bioreachable molecule to provide biorthogonal reaction chemistry.

### DETAILED DESCRIPTION

The present disclosure relates to compositions derived from bioreachable molecules, such as amino acids or hydroxy acids obtained from microbes (e.g., engineered microbes to overexpress desired biomolecules). Such bioreachable molecules can be reacted to form a cyclic dimer, which can be further chemically functionalized to provide a cyclic derivative. Such functionalization can include, e.g., inclusion of one or more reactive moieties, polymerizable moieties, or others. In turn, such cyclic derivatives can be employed as a monomer, a polymer, or a copolymer.

In one aspect, the cyclic derivative can include a structure having formula (I), (Ia), (Ib), or (Ic):

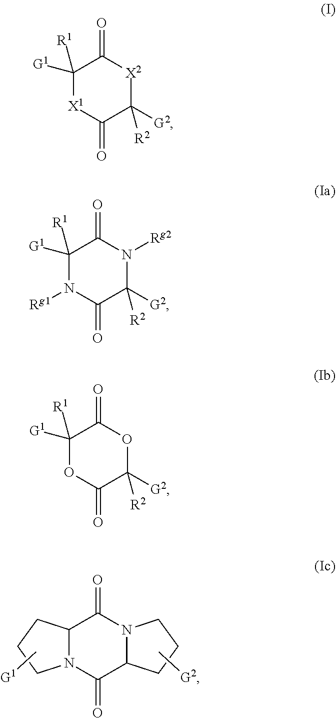

or a salt thereof. In some embodiments, each of G¹ and G² is or includes, independently, hydroxyl, carboxyl, amino, amido, cyanato, isocyanato, cyano, isocyano, optionally substituted alkenyl, optionally substituted alkynyl, optionally substituted (hetero)cycloalkyl, or optionally substituted epoxy. In some embodiments, each of R¹ and R² is, independently, H or optionally substituted alkyl. In other embodiments, X¹ is oxy or —N—R^{g1}, and X² is oxy or —N—R^{g2}. In yet other embodiments, each of R^{g1} and R^{g2} is, independently, H, optionally substituted alkyl, optionally substituted aryl, or optionally substituted aralkyl. In particular embodiments, R^{g1} and G¹, taken together with the nitrogen to which R^{g1} is bound, and/or R^{g2} and G², taken together with the nitrogen to which R^{g2} is bound, can optionally form an optionally substituted heterocyclyl.

In another aspect, the cyclic derivative can include a structure having formula (II):

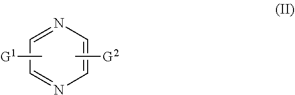

or a salt thereof, wherein each of G¹ and G² comprises, independently, hydroxyl, carboxyl, amino, amido, cyanato, isocyanato, cyano, isocyano, optionally substituted alkenyl, optionally substituted alkynyl, optionally substituted (hetero)cycloalkyl, or optionally substituted epoxy.

As can be seen, in some instances, G¹ and G² include one or more reactive moieties, which in turn can provide a polymer when the cyclic derivative is employed as a monomer. Within a polymer, the same cyclic derivative can be employed, or two or more different cyclic derivatives may be employed. Illustrative reactive moieties include, e.g., those described herein for R^{G}, R^{G1}, or R^{G2}, such as hydroxyl, halo, haloalkyl, carboxyl, amino, amido, cyanato, isocyanato, cyano, isocyano, optionally substituted alkyl, optionally substituted alkenyl, optionally substituted alkynyl, optionally substituted (hetero)cycloalkyl (e.g., optionally substituted oxiranyl or optionally substituted oxetanyl), or optionally substituted epoxy.

In some embodiments, each of G¹ and G² has a structure of:

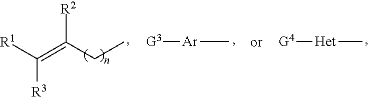

in which each of R¹, R², and R³ is, independently, H, optionally substituted alkyl, or optionally substituted alkenyl; Ar is optionally substituted arylene, such as divalent forms of benzene, naphthalene, biphenyl, phenoxy, aniline, etc. (boiler plate here would be great); Het is optionally substituted heterocyclyldiyl, such as divalent forms of indole, benzofuran, thianaphthene, imidazole, furan, thiophene; and each of G³ and G⁴ can be optionally substituted alkenyl (e.g., vinyl, allyl, homoallyl, olefin, and combinations thereof, as well as any described herein). In the foregoing structure, n can be an integer selected from 1 through 5.

In yet other embodiments, each of G¹ and G² has a structure of:

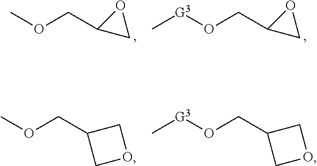

OH, or -G³OH, in which G³ can be optionally substituted alkylene, optionally substituted arylene, or optionally substituted (aryl)(alkyl)ene. In one embodiment, G³ can be methylene, ethylene, n-propylene, isopropylene, n-butylene, 2-methylpropylene, n-pentylene, 2-methylbutylene, 2,3-dimethylpropylene, 1,4-phenylene, methylene-phenylene, para-methylene-phenylene, ethylene-phenylene, or para-ethylene-phenylene.

In some embodiments, each of G¹ and G² can include one or more linkers (e.g., L^{G1}, L^{G2}, L^{G3}, L^{G4}, Ar^{G1}, Ar^{G2}, Het^{G1}, or Het^{G2}) attached to a reactive moiety (e.g., R^{G}, R^{G1}, or R^{G2}). Illustrative linkers include, e.g., a covalent bond, an amide bond, —NR^{N1}— (in which R^{N1} is H or optionally substituted alkyl), oxy, optionally substituted alkylene, optionally substituted alkenylene, optionally substituted heteroalkylene, optionally substituted arylene, optionally substituted (aryl)(alkyl)ene, optionally substituted heterocyclyldiyl, or optionally substituted (heterocyclyl)(alkyl)ene, as well as combinations thereof. Yet other linkers can include -L^{G1}-L^{G3}-, -L^{G1}-Ar^{G1}—, -L^{G1}-Het^{G1}-, -L^{G1}-Ar^{G1}-L^{G3}-, -L^{G2}-L^{G4}-, -L^{G2}-Ar^{G2}—, -L^{G2}-Het^{G2}-, or -L^{G2}-Ar^{G2}-L^{G4}-, for any L^{G1}, L^{G2}, L^{G3}, L^{G4}, Ar^{G1}, Ar^{G2}, Het^{G1}, or Het^{G2} described herein.

In particular embodiments, the cyclic derivative can include a structure having formula (Id):

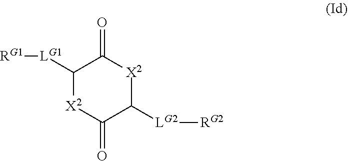

or a salt thereof, in which X¹ and X² can be any described herein; L^{G1} and L^{G2} can be any linker described herein; and R^{G1} and R^{G2} can be any reactive moiety described herein.

Such linkers and reactive moieties may be attached to a side chain present in the amino acid or hydroxy acid employed to form the cyclic dimer. Exemplary side chains can include, e.g., alkyl, amidoalkyl, aminoalkyl, carboxyalkyl, hydroxyalkyl, phenyl, aryl, aralkyl, hydroxyphenyl, hydroxyaryl, hydroxyaralkyl, or heterocyclyl. Accordingly, each of G¹ and G² can include any of such side chains that has been reacted to provide a linker (e.g., L^{G1}, LG², L^{G3}, L^{G4}, Ar^{G1}, Ar^{G2}, Het^{G1}, or Het^{G2}) attached to a reactive moiety (e.g., R^{G}, R^{G1}, or R^{G2}).

In other embodiments, the cyclic derivative can include a structure having formula (Ie), (If), or (Ig):

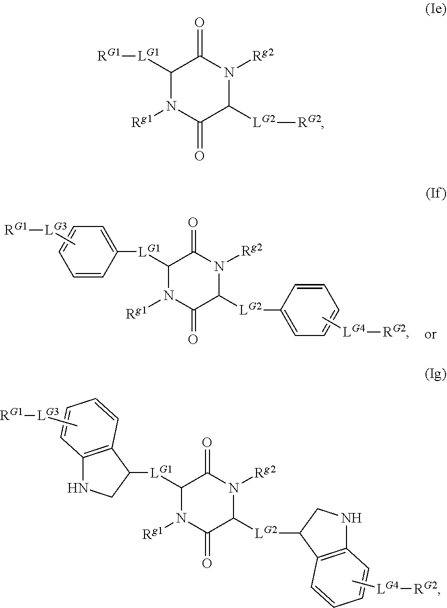

or a salt thereof, in which R^{g1} and R^{g2} can be any described herein; L^{G1}, L^{G2}, L^{G3}, and L^{G4} can be any linker described herein; and R^{G1} and R^{G2} can be any reactive moiety described herein.

In yet other embodiments, the cyclic derivative can include a structure having formula (Ih), (Ii), or (Ij):

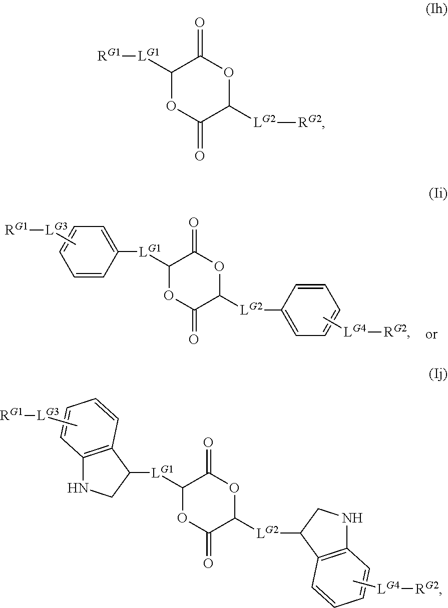

or a salt thereof, in which L^{G1}, L^{G2}, L^{G3}, and L^{G4} can be any linker described herein; and R^{G1} and R^{G2} can be any reactive moiety described herein.

In some embodiments (e.g., in formula (Ie), (If), (Ig), (Ih), (Ii), (Ij), or any herein), each of L^{G1}, L^{G2}, L^{G3}, and L^{G4} is, independently, a covalent bond, oxy, optionally substituted alkylene, or optionally substituted heteroalkylene. In particular embodiments, the optionally substituted heteroalkylene is —O-Ak- or -Ak-O—, in which Ak is an optionally substituted alkylene (e.g., C₁₋₃ alkylene). In other embodiments, each of R^{G1} and R^{G2} is, independently, optionally substituted alkylene (e.g., vinyl, allyl, optionally substituted butenyl, optionally substituted pentenyl, and the like), optionally substituted (hetero)cycloalkyl (e.g., optionally substituted epoxy, optionally substituted oxiranyl, optionally substituted oxetanyl, and the like).

In one embodiment, the reactive moiety is or includes an optionally substituted alkenyl or an optionally substituted epoxy. Thus, G¹, G², R^{G}, R^{G1}, or R^{G2} can include such a reactive moiety. In some embodiments, the optionally substituted alkenyl has a structure of:

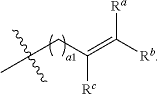

In other embodiments, the optionally substituted epoxy has a structure of:

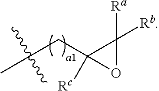

In each of these structures, each of R^{a}, R^{b}, and R^{c} is, independently, H, optionally substituted alkyl, or optionally substituted alkenyl; and a1 is an integer of from 0 to 4.

In another embodiment, the reactive moiety is or includes hydroxyl, optionally substituted hydroxyalkyl, or optionally substituted hydroxyaryl. In particular embodiments, the cyclic derivative can include a structure selected from the group of:

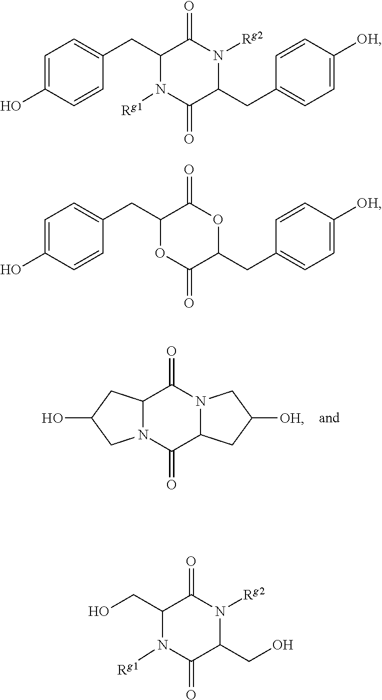

or a salt thereof, in which R^{g1} and R^{g2} can be any described herein.

Reactive moieties can also be characterized as a polymerizable group. A polymerizable group includes groups that form homopolymers or copolymers. In a first embodiment, the polymerizable group can form predominately homopolymers, meaning that the compound A forms polymers symbolized as -(A-A-A)ₓ-, wherein x is an integer. These groups are defined as homopolymerizable. Examples of such groups are unsaturated groups, such as vinyl and allyl groups, oxiranes (ethylene oxides or epoxides), aziridines (ethylene imines), oxetanes. In another embodiment, the polymerizable group is copolymerizable, i.e., a second compound B is required to form polymers -(A-B-A-B)ₓ-, wherein x is an integer. Examples of such groups are carboxylic acids, hydroxyl groups, amino groups, thiol groups; and examples for the respective copolymer monomer would be diols or diamines, diacids, diacid anhydrides, isocyanates, di-isocyanates.

In one embodiment, the polymerizable group can be selected from a vinyl group, an allyl group, an epoxy group, or a combination thereof.

In another embodiment, at least 35 wt. %, such as at least 40 wt. %, at least 45 wt. %, at least 50 wt. %, at least 55 wt. %, at least 60 wt. %, at least 65 wt. %, at least 70 wt. %, at least 75 wt. %, at least 80 wt. %, or at least 85 wt. % of the compound or the composition is comprised by the moiety. In another embodiment, not more than 98 wt. %, such as not more than 96 wt. %, not more than 95 wt. %, not more than 94 wt. %, not more than 92 wt. %, or not more than 90 wt. % of the compound or the composition are comprised by the moiety. In yet one further embodiment, the moiety of the compound or the composition has weight percentage in the range between 30 wt. % to 99.5 wt. %, such as 40 wt. % to 98 wt. %, or even 50.5 wt. % to 96 wt. %.

In yet one further embodiment, at least 60 wt. %, at least 65 wt. %, at least 70 wt. %, at least 75 wt. %, at least 80 wt. %, at least 85 wt. %, or at least 88 wt. % of the compound or the composition are comprised by the sum of weight percentages of the moiety and the polymerizable group. In another embodiment, not more than 99.9 wt. %, such as not more than 99 wt. %, not more than 98 wt. %, not more than 96 wt. %, not more than 94 wt. %, not more than 92 wt. %, not more than 90 wt. %, not more than 85 wt. %, or not more than 80 wt. % of the compound or the composition are comprised by the sum of weight percentages of the moiety and the polymerizable group. In yet one further embodiment, the sum of weight percentages of the moiety and the polymerizable group can range between 55 wt. % to 99.99 wt. %, such as 65 wt. % to 99 wt. %, or 75 wt. % to 98 wt. %.

In any of the formulas herein, R^{g1} and R^{g2} can be H, optionally substituted alkyl, haloalkyl, alkoxyalkyl, or any combination thereof. Other non-limiting R^{g1} and R^{g2} groups include, independently for each occasion, hydrogen or C₁₋₂₀ straight or branched alkyl chains, such as methyl, ethyl, n-propyl, 2-propyl, 1-butyl, 2-butyl, 2-methylpropyl, pentyl, 2-methylbutyl, 2,2-dimethylpropyl, hexyl, 2-methylpentyl, 3-methylpentyl, 2,2-dimethylbutyl, 2,3-dimethylbutyl, heptyl, 2-methylhexyl, 3-methylhexyl, 2,2-dimethylpentyl, 2,3-dimethylpentyl, 2,4-dimethylpentyl, 3,3-dimethylpentyl, 3-ethylpentyl, 2,2,3-trimethylbutyl, octyl, 2-methylheptyl, 3-methylheptyl, 4-methylheptyl, 5-methylheptyl, 6-methylheptyl, 2-ethylhexyl, 3-ethylhexyl, 4-ethylhexyl, 2,3-dimethylhexyl, 2,4-dimethylhexyl, 2,5-dimethylhexyl, 3,4-dimethylhexyl, 3,5-dimethylhexyl, 4,5-dimethylhexyl, 2-propylpentyl, nonyl, decyl, undecyl, dodecyl, tridecyl, tetradecyl, pentadecyl, hexadecyl, heptadecyl, octadecyl, nonadecyl, and icosyl.

In a further embodiment, the foregoing compound or composition has a bio-based carbon content of at least 10%, such as at least 15%, at least 20%, at least 25%, at least 30%, or at least 35% as determined by ASTM D6866. Bio-based carbon content as defined herein is the percentage of carbons from renewable or biogenic sources, such as plants or animals over the total number of carbons in the compound.

For example, the following cyclic derivative is prepared from bio-sourced tyrosine and petro chemically epichlorohydrin:

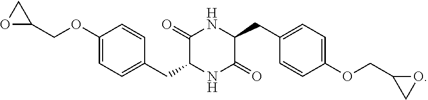 [SMILES: O=C1N[C@H](Cc2ccc(OCC3CO3)cc2)C(=O)N[C@H]1Cc1ccc(OCC2CO2)cc1]

Then, 16 carbon atoms are bio-based and 6 carbon atoms are petrochemically sourced. Upon analysis according to ASTM D6866, this compound has a bio-based carbon content of 16/(16+6)=72.7%.

Additional cyclic dimers and cyclic derivatives are provided below in Table 1.

TABLE 1
Non-limiting cyclic dimers and cyclic derivatives

| Compound No. | Structure |
|---|---|
| I-1 | 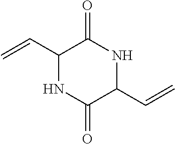 |
| I-2 | 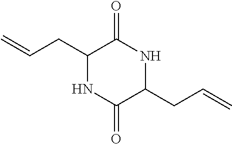 |
| I-3 | 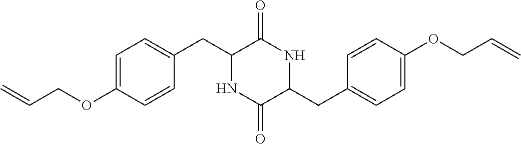 [SMILES: C=CCOc1ccc(CC2NC(=O)C(Cc3ccc(OCC=C)cc3)NC2=O)cc1] |
| I-4 | 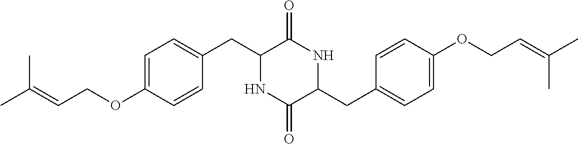 [SMILES: CC(C)=CCOc1ccc(CC2NC(=O)C(Cc3ccc(OCC=C(C)C)cc3)NC2=O)cc1] |
| I-5 | 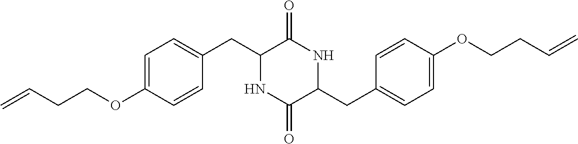 [SMILES: C=CCCOc1ccc(CC2NC(=O)C(Cc3ccc(OCCC=C)cc3)NC2=O)cc1] |
| I-6 | 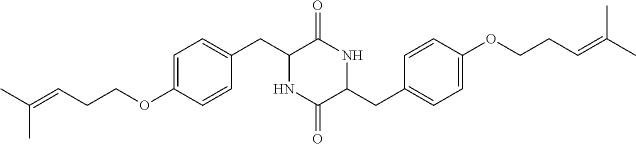 [SMILES: CC(C)=CCCOc1ccc(CC2NC(=O)C(Cc3ccc(OCCC=C(C)C)cc3)NC2=O)cc1] |
| I-7 | 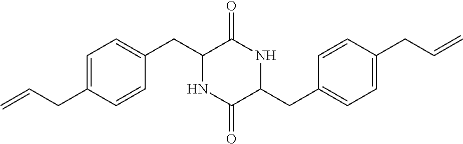 [SMILES: C=CCc1ccc(CC2NC(=O)C(Cc3ccc(CC=C)cc3)NC2=O)cc1] |
| I-8 | 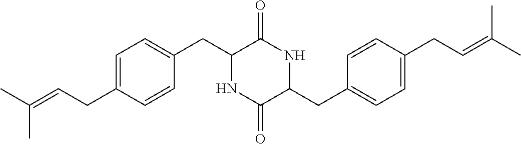 [SMILES: CC(C)=CCc1ccc(CC2NC(=O)C(Cc3ccc(CC=C(C)C)cc3)NC2=O)cc1] |
| I-9 | 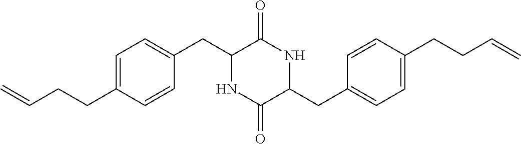 [SMILES: C=CCCc1ccc(CC2NC(=O)C(Cc3ccc(CCC=C)cc3)NC2=O)cc1] |
| I-10 | 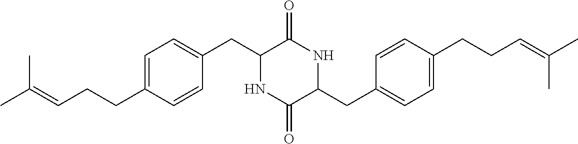 [SMILES: CC(C)=CCCc1ccc(CC2NC(=O)C(Cc3ccc(CCC=C(C)C)cc3)NC2=O)cc1] |
| I-11 | 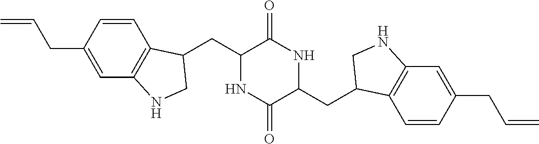 [SMILES: C=CCc1ccc2c(c1)NCC2CC1NC(=O)C(CC2CNc3cc(CC=C)ccc32)NC1=O] |
| I-12 | 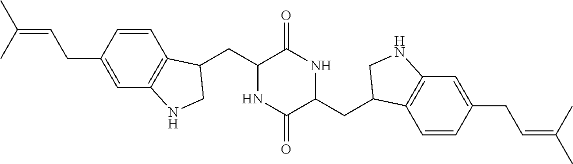 [SMILES: CC(C)=CCc1ccc2c(c1)NCC2CC1NC(=O)C(CC2CNc3cc(CC=C(C)C)ccc32)NC1=O] |
| I-13 | 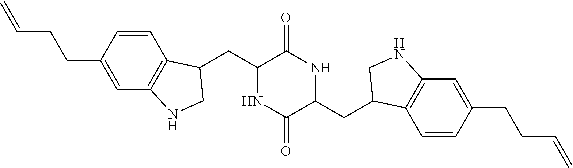 [SMILES: C=CCCc1ccc2c(c1)NCC2CC1NC(=O)C(CC2CNc3cc(CCC=C)ccc32)NC1=O] |
| I-14 | 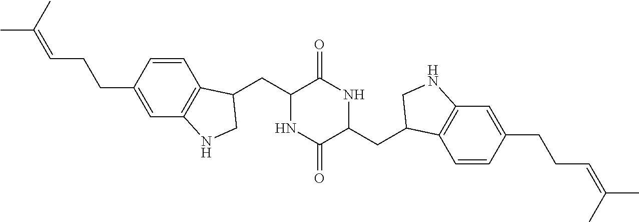 [SMILES: CC(C)=CCCc1ccc2c(c1)NCC2CC1NC(=O)C(CC2CNc3cc(CCC=C(C)C)ccc32)NC1=O] |
| I-15 | 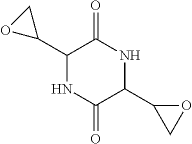 [SMILES: O=C1NC(C2CO2)C(=O)NC1C1CO1] |
| I-16 | 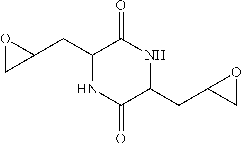 [SMILES: O=C1NC(CC2CO2)C(=O)NC1CC1CO1] |
| I-17 | 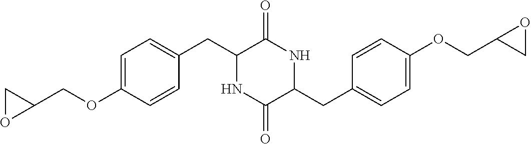 [SMILES: O=C1NC(Cc2ccc(OCC3CO3)cc2)C(=O)NC1Cc1ccc(OCC2CO2)cc1] |
| I-18 | 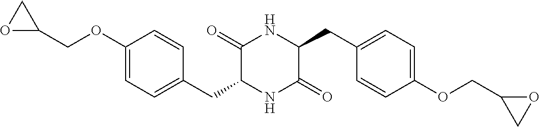 [SMILES: O=C1N[C@H](Cc2ccc(OCC3CO3)cc2)C(=O)N[C@H]1Cc1ccc(OCC2CO2)cc1] |
| I-19 | 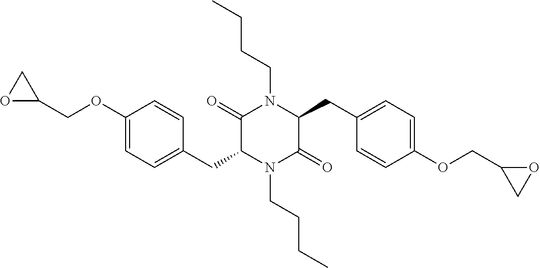 [SMILES: CCCCN1C(=O)[C@H](Cc2ccc(OCC3CO3)cc2)N(CCCC)C(=O)[C@H]1Cc1ccc(OCC2CO2)cc1] |
| I-20 | 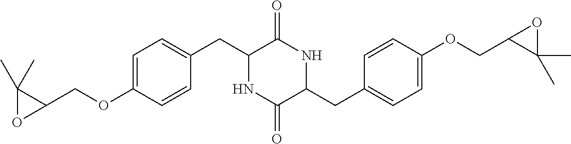 |
| I-21 | 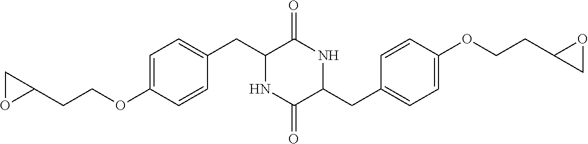 [SMILES: O=C1NC(Cc2ccc(OCCC3CO3)cc2)C(=O)NC1Cc1ccc(OCCC2CO2)cc1] |
| I-22 | 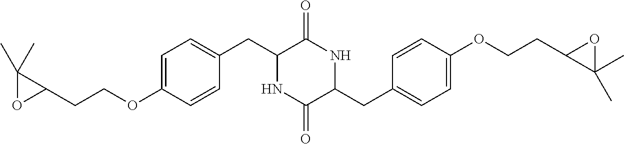 |
| I-23 | 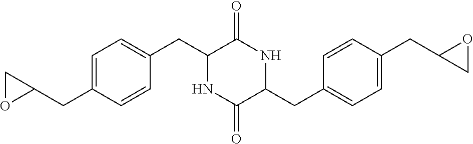 [SMILES: O=C1NC(Cc2ccc(CC3CO3)cc2)C(=O)NC1Cc1ccc(CC2CO2)cc1] |
| I-24 | 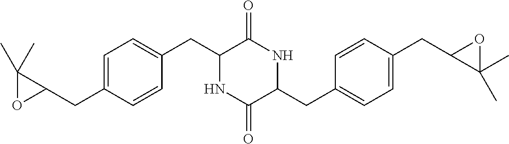 |
| I-25 | 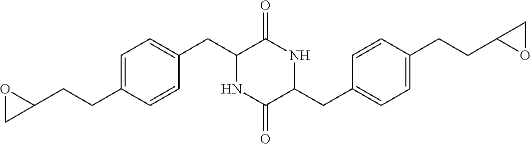 [SMILES: O=C1NC(Cc2ccc(CCC3CO3)cc2)C(=O)NC1Cc1ccc(CCC2CO2)cc1] |
| I-26 | 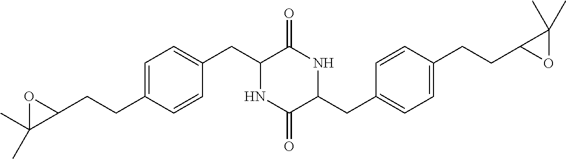 |
| I-27 | 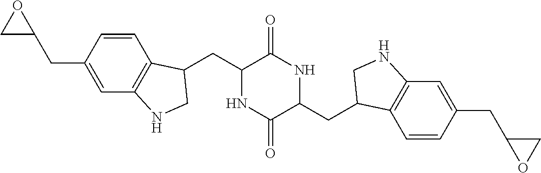 [SMILES: O=C1NC(CC2CNc3cc(CC4CO4)ccc32)C(=O)NC1CC1CNc2cc(CC3CO3)ccc21] |
| I-28 | 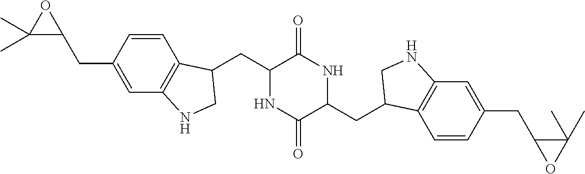 |
| I-29 | 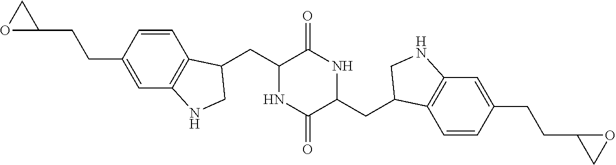 [SMILES: O=C1NC(CC2CNc3cc(CCC4CO4)ccc32)C(=O)NC1CC1CNc2cc(CCC3CO3)ccc21] |
| I-30 | 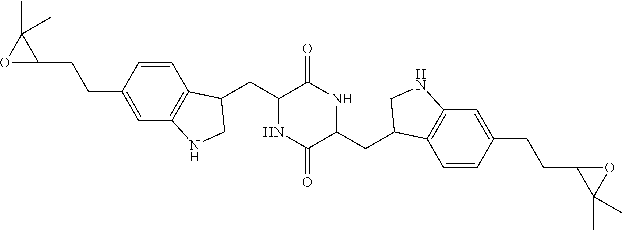 [SMILES: CC1(C)OC1CCc1ccc2c(c1)NCC2CC1NC(=O)C(CC2CNc3cc(CCC4OC4(C)C)ccc32)NC1=O] |

The cyclic derivatives herein can be prepared in any useful manner, such as by providing a first biomolecule and a second biomolecule and forming a dimer between the first and second biomolecules. The first and second biomolecules can be any herein, including, e.g., amino acids, hydroxy acids, hydroxymandelic acid, hydroxyproline, serine, tyrosine, tryptophan, phenylalanine, vinylglycine, allylglycine, and derivatives of any of these including an optionally substituted alkenyl. Additional non-limiting biomolecules are further described herein. The dimer can be further functionalized (e.g., to include one or more linkers and/or reactive moieties). The methods herein can further include epoxidizing the dimer in the presence of an oxidant (e.g., chlorine, hypochlorous acid, a peroxycarboxylic acid, a peroxycarboxylate, a peroxyphthalate, or a combination thereof).

In other embodiments, the cyclic derivates are prepared by providing an organism with a plurality of amino acids, thereby producing a plurality of prenylated amino acids; and then forming a dimer between two of the plurality of amino acids. The plurality of amino acids can be any herein, including, e.g., glycine, serine, tyrosine, tryptophan, phenylalanine, and the like. Additional amino acids are described herein.

In yet other embodiments, the cyclic derivatives herein can be prepared by processes analogous to those established in the art, for example, by the reaction sequences shown in Schemes 1-3.

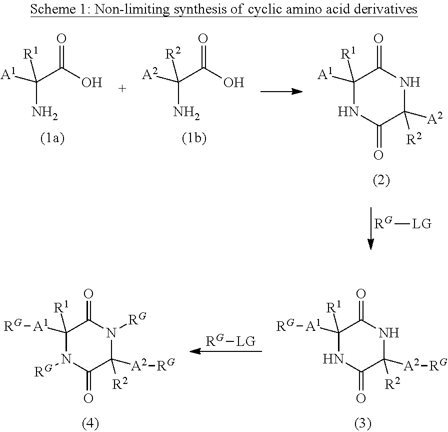

As seen in Scheme 1, amino acids (1a, 1b) can be provided, in which R¹ and R² can be H, alkyl, any described herein for R¹ and R²; and in which A¹ and A² can be an amino acid side chain or a functionalized form thereof. Substituents within the amino acid can be optionally protected with a protecting group (e.g., an N-protecting group for amino or an O-protecting group for hydroxyl) or can be optionally functionalized to provide a better leaving group (e.g., an alkylating agent for oxygen to provide an alkoxy leaving group). Amino acids (1a, 1b) can be the same or different. Furthermore, such amino acids can be optionally provided by a biological resource.

Cyclic amino acids (2) can be provided by dimerization and cyclization of the amino acids (1a, 1b) in the presence of a solvent (e.g., ethylene glycol). If desired, dimers can first be formed to promote internal cyclization within the dimer. Dimerization can be performed in any useful manner (e.g., with use of protecting groups); and subsequent cyclization can optionally be performed under catalytic conditions (e.g., with subsequent deprotection chemistry to remove protecting groups).

Reactive moieties can then be provided. Cyclic amino acid (2) can be functionalized with R^{G}-LG to provide a cyclic derivative (3), in which R^{G} is or includes a reactive moiety (e.g., any described herein, such as for R^{G}, R^{G1}, or R^{G2}) and LG is a leaving group (e.g., halo).

Further functionalization of compound (3) can provide another cyclic derivative (4), in which nitrogen atoms of the diketopiperazine can include be further substituted. Here, compound (3) can be functionalized with R^{g}-LG to provide cyclic derivative (4), in which R^{g} can be any described herein (e.g., such as for R^{g1} and R^{g2}).

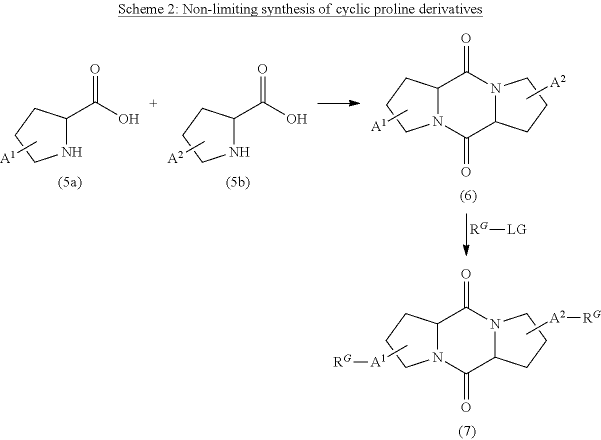

As seen in Scheme 2, proline derivatives (5a, 5b) can be provided, in which A¹ and A² can be an amino acid side chain or a functionalized form thereof. In one instance, A¹ and A² includes hydroxyl for a hydroxyproline derivative (e.g., 4-hydroxyproline). Amino acids (5a, 5b) can be the same or different and can be optionally provided by a biological resource.

Cyclic amino acids (6) can be provided by dimerization and cyclization of the amino acids (5a, 5b) in the presence of a solvent (e.g., ethylene glycol). Reactive moieties can then be provided by functionalizing the cyclic amino acid (6) with R^{G}-LG to provide a cyclic derivative (7), in which R^{G} is or includes a reactive moiety (e.g., any described herein, such as for R^{G}, R^{G1}, or R^{G2}) and LG is a leaving group (e.g., halo).

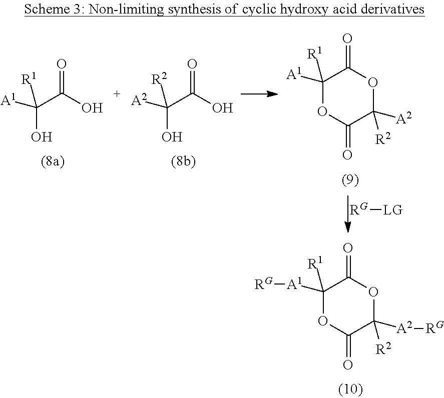

Hydroxy acids can also be employed to form cyclic derivatives. As seen in Scheme 3, hydroxy acids (8a, 8b) can be provided, in which R¹ and R² can be H or alkyl; and in which A¹ and A² can be alkyl, aryl, aralkyl, or a substituted form thereof. Substituents within the hydroxy acid can be optionally protected with a protecting group (e.g., an O-protecting group for hydroxyl) or can be optionally functionalized to provide a better leaving group (e.g., an alkylating agent for oxygen to provide an alkoxy leaving group). Hydroxy acids (8a, 8b) can be the same or different and can be optionally provided by a biological resource.

Cyclic hydroxy acids (9) can be provided by dimerization and cyclization of the hydroxy acids (8a, 8b) in the presence of a solvent, and further functionalization can include use of R^{G}-LG to provide a cyclic derivative (10), in which R^{G} is or includes a reactive moiety (e.g., any described herein, such as for R^{G}, GR or R^{G2}) and LG is a leaving group (e.g., halo).

Methods herein also include those for preparing a resin, which can include reacting a cyclic dimer or a cyclic derivative with a reagent. The cyclic dimer or cyclic derivative can include, e.g., OH groups. Furthermore, reacting can include initiation at a ratio of moles of OH groups per moles of reagent ranging from 10:1 to 1:1. Non-limiting reagents include, e.g., epichlorohydrin, epibromohydrin, allyl halides, vinyl halides, unsaturated acids, allyl halides, vinyl halides, unsaturated acids, or any combination thereof.

In some instances, providing a reactive moiety by way of the reagent can further result in polymerization of cyclic derivatives. For instance, as shown below:

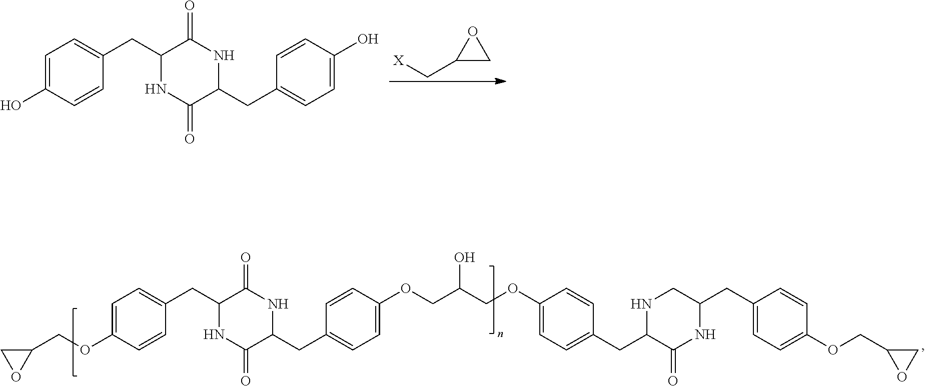

wherein X can be a leaving group (e.g., halo, such as Cl or Br).

Methods of preparing a resin can further include adding an oxidant. In this instance, the cyclic dimer or cyclic derivative can be reacted with a reagent to provide a reactive group (e.g., a polymerizable group) that can be further treated with an oxidant, such as by an epoxidation reaction:

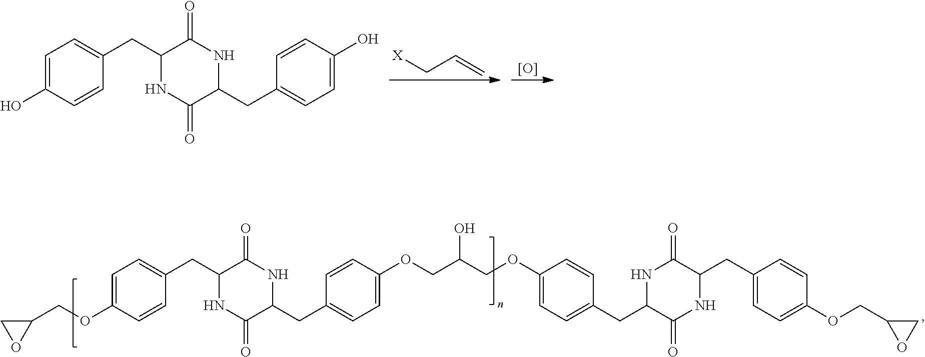

wherein X is a leaving group (e.g., halo, such as Cl or Br), [O] is an oxidant, and n is an integer including zero. The epoxidation reaction can be stoichiometric, i.e., one mole of epichlorohydrin or epibromohydrin per mole of hydroxy groups in the moiety. Alternatively, epoxidation can be conducted to a lesser degree, wherein the ratio of moles of hydroxy group over moles of reagent can range from 20:1 to 0.9:1, such as from 15:1 to 1:1, 10:1 to 1:1, or 5:1 to 1:1. This is true for any other reagent that renders the moiety polymerizable, such as allyl halides or vinyl halides.

The oxidation reaction in the above scheme serves to render epoxides from unsaturated organic groups. In one embodiment, an oxidation reaction is omitted to allow the unsaturated group to be the polymerizable group. Here too, all hydroxyl groups or a fraction thereof can react to give a polymerizable group. Oxidants can be peroxides, percarboxylic acids, percarboxylic esters, peroxycarboxylates, peroxyphthalates, percarboxylic salts, chlorine, hypochlorous acid, hypochlorites, or combinations thereof. In epoxidized dimers, the epoxy groups can be symmetrically located in ortho, meta, or para positions, but also asymmetrical locations, i.e., ortho-meta, ortho-para, or meta-para are contemplated within this disclosure.

### Biomolecules, Including Amino Acids, Hydroxy Acids, and Derivatives Thereof

As described herein, the compositions and methods herein can employ biomolecules, which can be further undergo dimerization, cyclization, and/or functionalization. Non-limiting biomolecules include amino acids and hydroxy acids (e.g., alpha hydroxy acids), such as glycine, vinylglycine, allylglycine, alkenylglycine, tyrosine, O-allyltyrosine, O-alkenyltryrosine, tryptophan, allyltryptophan, alkenyltryptophan, phenylalanine, allylphenylalanine, alkenylphenylalanine, hydroxymandelic acid (e.g., 4-hydroxymandelic acid, 3-hydroxymandelic acid, DL-4-hydroxy-3-methoxymandelic acid, DL-3,4-dihydroxymandelic acid, and others), hydroxyproline (e.g., 4-hydroxyproline), or serine.

Yet other non-limiting biomolecules can include, e.g., derivatives of any amino acids or hydroxy acids including an alkenyl, alkenyloxy (e.g., —O-Ak, in which Ak is alkenyl), carboxyl, or hydroxyl moiety. In particular embodiments, the biomolecule is an L-amino acid or a functionalized L-amino acid having an alkenyl, alkenyloxy (e.g., —O-Ak, in which Ak is alkenyl), carboxyl, or hydroxyl moiety.

Other non-limiting biomolecules also include the following:

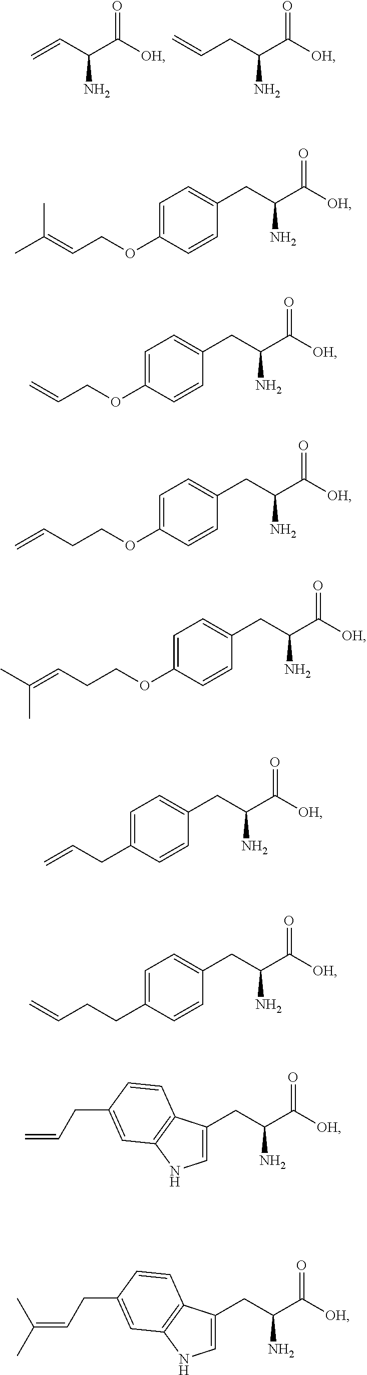

as well as salts thereof and stereoisomers thereof.

Biomolecules can be formed in any useful manner. In one instance, the biomolecules are produced from yeast, gram positive bacteria, gram negative bacteria, or fungi. In other embodiments, amino acids, as well as derivatives thereof and/or dimers thereof, are produced biologically by way of fermentation and/or prenylation. In particular embodiments, prenylation may involve feeding the organism the starting amino acid. In yet other embodiments, amino acid dimers are produced by chemical means using petro-based starting materials.

### Applications

The compositions herein can be employed as ingredients and/or monomers in any useful application. Exemplary, non-limiting applications include adhesives, coatings, films, and plastics. Such applications can include materials for use in constructing electronics, industrial adhesives, architectural adhesives and coatings, civil engineering adhesives and coatings, transportation adhesives and coatings, handheld devices, electronic devices, energy storage devices, energy generation devices, personal electronics (e.g., smart phones, laptops, or tablets), displays, sensors, semi-conductor materials (e.g., such as in chip patterning, manufacturing, and packaging), packages, and the like.

Yet other applications include use of the composition as a polymer curative, a resin (e.g., an ion free resin), a monomer for a polymer or a copolymer, and the like. The composition can be provided in any useful form, such as a film, a composite structure, a bulk structure, a fiber, or a particle. The composition can optionally include one or more hardeners for use with the cyclic derivatives. Non-limiting hardeners include, e.g., diamines (such as 1,4-diamino butane (DAB) or 1,13-diamino-4,7,10-trioxatridecane (TDD). If desired, the composition can also include an accelerator, such as tris(dimethylaminomethyl)phenol, or other additives (e.g., resorcinol diglycidyl ether).

In particular embodiments, the compositions herein can undergo bio-triggered degradation for debonding of adhesives, coatings, and composites. Degradation can be triggered, e.g., by employing one or more proteases, hydrolases, and the like.

In some embodiments, the present disclosure encompasses methods for manufacturing any use herein (e.g., an adhesive, a coating, a film, a plastic, a composite, an electronic device, an energy storage device, an energy generation device, and the like) by applying a composition herein (e.g., any foregoing compound) in the assembly of the adhesive, the coating, the film, the plastic, the composite, the electronic device, the energy storage device, or the energy generation device. In other embodiments, the composition is provided as a polymer curative.

### EXAMPLES

### Example 1: Copolymers from Amino Acid Dimers and Hydroxy Acid Dimers

The composition herein can be employed to provide a copolymer. In one instance, amino acids or hydroxy acids are employed to provide cyclic derivatives, which can be further functionalized with polymerizable moieties. Exemplary polymerizable moieties include, e.g., hydroxyl, halo, amino, cyanato, isocyanato, cyano, isocyano, optionally substituted alkenyl, optionally substituted alkynyl, optionally substituted (hetero)cycloalkyl, or optionally substituted epoxy groups.

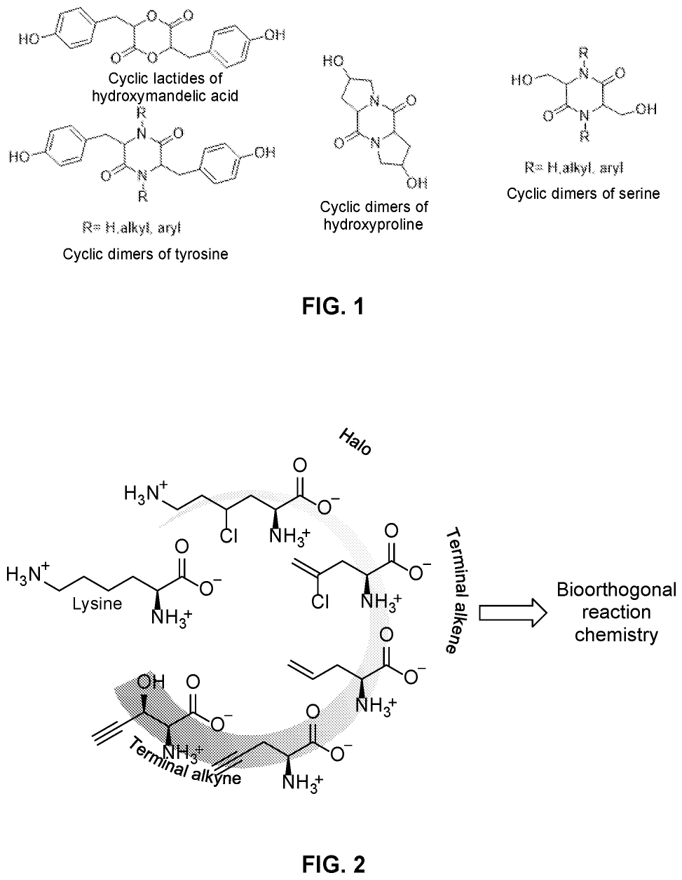

FIG. 1 shows exemplary cyclic derivatives, including a cyclic lactide formed from hydroxymandelic acid (e.g., 4-hydroxymandelic acid), a cyclic dimer formed from tyrosine, a cyclic dimer formed from hydroxyproline (e.g., 4-hydroxyproline), and a cyclic dimer formed from serine. Amino acids, when employed, can have any useful stereochemistry (e.g., L- or D-amino acids). As seen in FIG. 2, bioreachable molecules, such as amino acids, can be further functionalized to provide polymerizable moieties, such as halo, alkenyl, and alkenyl groups.

### Example 2: Ion-Free Resins from Amino Acids

The compositions herein can be employed to provide an ion-free resin. For instance, amino acids can be employed to produce cyclic derivatives, which can be further functionalized with one or more unsaturated alkyl moieties, such as vinyl, allyl, or homoallyl moieties attached to a side chain of the amino acid. These unsaturated alkyl moieties can then be epoxidized with an oxidation reagent, thereby providing a cyclic ether group. In particular, these reactions can be conducted to minimize ion content, which can provide higher purity monomers and polymers.

Such ion-free resins (e.g., having a total ion content less than about 1 part per thousand) can be employed as an ingredient or a monomer in any useful composition or material. Illustrative compositions and materials include, e.g., coatings, adhesives, films, and plastics in the construction of electronics, industrial adhesives, architectural adhesives and coatings, civil engineering adhesives and coatings, transportation adhesives and coatings, handheld devices, smart phones, laptops, tablets, displays, sensors, semi-conductor chip patterning, manufacturing, and packaging.

### Example 3: Non-Limiting Synthesis of Tyrosine Dimer

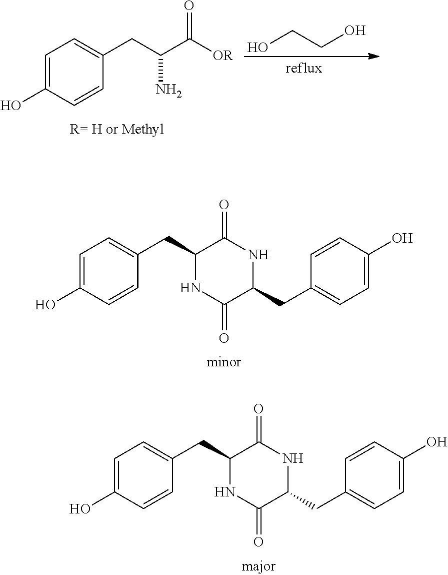

In a 3 L two-neck round bottom flask equipped with magnetic stirrer and overhead condenser, 200 g of Tyr-OH and 800 ml of ethylene glycol were mixed, and the flask was placed in silicon oil bath. The oil bath was heated to 190° C., and the reaction mixture was stirred for 7 hours (h). The conversion of starting material was followed up by HPLC. After 7 h, the reaction mixture was cooled down to room temperature, and the precipitated solid was filtered and washed with ethanol (2×200 ml). The solid was then dried in vacuum oven and used as is for the next step. (Yield: 64%)

### Example 4: Non-Limiting Synthesis of 4-Hydroxy-Proline Dimer

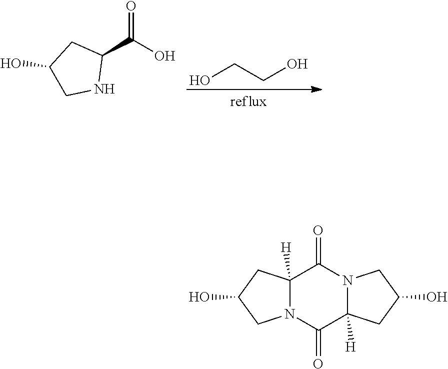

In a two-neck 1 L round bottom flask equipped with magnetic stirrer and overhead condenser, 100 g of trans-4-hydroxy-L-proline and 200 ml of ethylene glycol were mixed, and the flask was placed in silicon oil bath. The oil bath was heated to 190° C., and the reaction mixture was stirred for 7 h. After 7 h, the reaction mixture was cooled down to room temperature, and the precipitated solid was filtered and washed with acetone (2×100 ml). The solid was then dried in vacuum oven. (Yield: 44%, isolated 37.95 grams of product) NMR ¹H NMR (D₂O): 4.75 (d, 1H), 4.63 (d, 1H), 3.69 (d, 1H), 3.537 (d, 1H), 2.33 (d, 1H), 2.20 (d, 1H).

### Example 5: Non-Limiting Stepwise Synthesis of Tyrosine Dimer

The following route could be applicable for dimers from different amino acids.

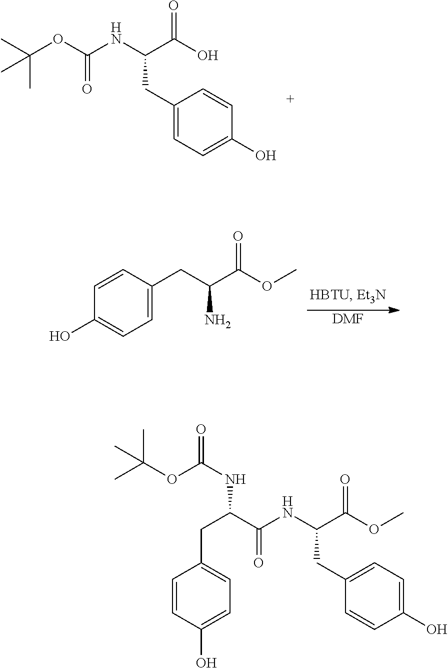

### Step 1: Preparation of (S)-methyl 2-((R)-2-((tert-butoxycarbonyl)amino)-3-(4-hydroxyphenyl) propanamido)-3-(4-hydroxyphenyl)propanoate

A 1 L reactor equipped with a magnetic stirrer, temperature probe, and nitrogen inlet was charged with ((S)-2-((tert-butoxycarbonyl)amino)-3-(4-hydroxyphenyl)propanoic acid (33.2 g, 118 mmol), (S)-methyl 2-amino-3-(4-hydroxyphenyl)propanoate (20 g, 102 mmol), hexafluorophosphate benzotriazole tetramethyl uronium (“HBTU,” 48.3 g, 127 mmol) and DMF (120 mL). The solution was stirred for 15 minutes and then cooled to 0° C. Triethylamine (42.6 mL, 306 mmol) was added to the mixture over 15 minutes. After the addition was completed, the cooling bath was removed, and the reaction was stirred overnight. After 18 h, the HPLC of the aliquot showed complete conversion of the starting materials. One hundred mL of water was slowly added to the reaction at 0° C. After stirring for 30 minutes (min), the mixture was diluted with EtOAc (150 mL), and the layers were separated. The organic layer was washed with aqueous sodium carbonate (10%, 3×50 mL) and finally with brine (50 mL). The organic layer was then dried over anhydrous sodium sulfate, filtered, and concentrated to dryness to afford the desired product as a thick oil. The product was used in the next step without further purification.

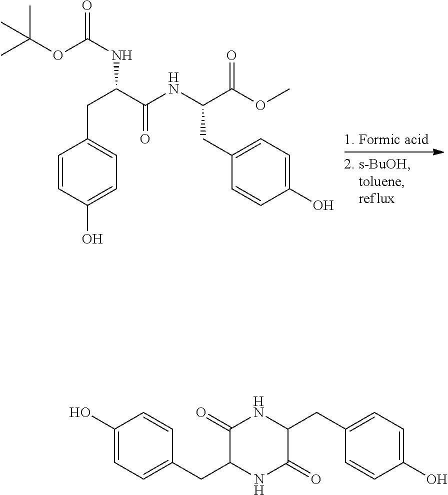 [SMILES: COC(=O)[C@H](Cc1ccc(O)cc1)NC(=O)[C@H](Cc1ccc(O)cc1)NC(=O)OC(C)(C)C ; O=C1NC(Cc2ccc(O)cc2)C(=O)NC1Cc1ccc(O)cc1]

### Step 2: Preparation of 3,6-bis(4-hydroxybenzyl)piperazine-2,5-dione

A 3 L single-neck reactor was charged with (S)-methyl 2-((R)-2-((tertbutoxycarbonyl)amino)-3-(4-hydroxyphenyl)propanamido)-3-(4-hydroxyphenyl)propanoate (42 g, 91.6 mmol) and formic acid (420 mL), the mixture was stirred at ambient temperature for 5 h, and the formic acid and s-butanol were removed under reduced pressure. The residue was dissolved in sec-butanol (1600 mL) and toluene (400 mL), and the solution was refluxed for 3 h. The reaction was monitored by HPLC and, after the reaction was completed, the reaction mixture was concentrated to yield the crude material as an off-white solid. The crude material was dissolved in 5% NaOH in water at 5° C. and extracted with 250 ml of ethyl acetate. The aqueous layer was acidified to pH 3 by the slow addition of 10% HCl (aq). The solid material was separated by filtration, washed with water, and dried under vacuum. The solid was suspended in 200 ml of acetonitrile and filtered again and dried to get a white solid as a pure product. (Yield: 22 g, 73%). NMR 1H NMR (DMSO): 9.20 (s, 1H), 7.76 (s, 1H), 6.84 (d, J=8.4 Hz, 2H), 6.67 (d, J=8.5 Hz, 2H), 3.85 (s, 1H), 2.55-2.51 (m, 1H), 2.12 (d, J=6.6 Hz, 1H).

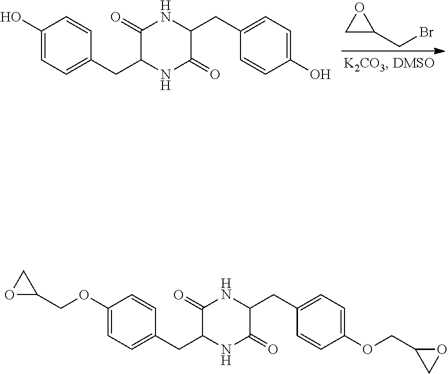 [SMILES: O=C1NC(Cc2ccc(O)cc2)C(=O)NC1Cc1ccc(O)cc1 ; BrCC1CO1 ; O=C1NC(Cc2ccc(OCC3CO3)cc2)C(=O)NC1Cc1ccc(OCC2CO2)cc1]

### Step 3: Preparation of 3,6-bis(4-(oxiran-2-ylmethoxy)benzyl)piperazine-2,5-dione

A 1 L single-neck reactor was charged with 3,6-bis(4-hydroxybenzyl)piperazine-2,5-dione (2 g, 6.13 mmol) and DMSO (30 mL), and the mixture was stirred at ambient temperature for 30 min in order to allow the starting materials to dissolve. Potassium carbonate (3.4 g, 24.52 mmol) was added. and the stirring was continued for 30 minutes. Epibromohydrin (1.6 mL, 18.40 mmol) was then added. and the reaction mixture was stirred for 2 days at room temperature. The reaction mixture was filtered to remove the solids and the solid was rinsed with DMSO (20 mL). The filtrate solution obtained was slowly poured into ice cold water (100 ml). The solid was filtered, washed with water (100 ml), and dried under vacuum. The solid was suspended in 120 ml of acetonitrile, filtered, and the solid was dried under vacuum to yield an off-white solid. (Yield: 1.9 g, 71%). NMR-GLC19575 ¹H NMR (DMSO): 7.86 (s, 1H), 6.95 (d, J=8.5 Hz, 2H), 6.87 (d, J=8.5 Hz, 2H), 4.31, 4.21 (m, 1H), 3.93 (s, 1H), 3.77 (dt, J=11.1, 6.2 Hz, 1H), 3.28 (d, J=2.6 Hz, 1H), 2.80 (t, J=4.6 Hz, 1H), 2.67 (s, 1H), 2.56 (dd, J=13.7, 4.4 Hz, 1H), 2.23 (dd, J=13.6, 6.0 Hz, 1H).

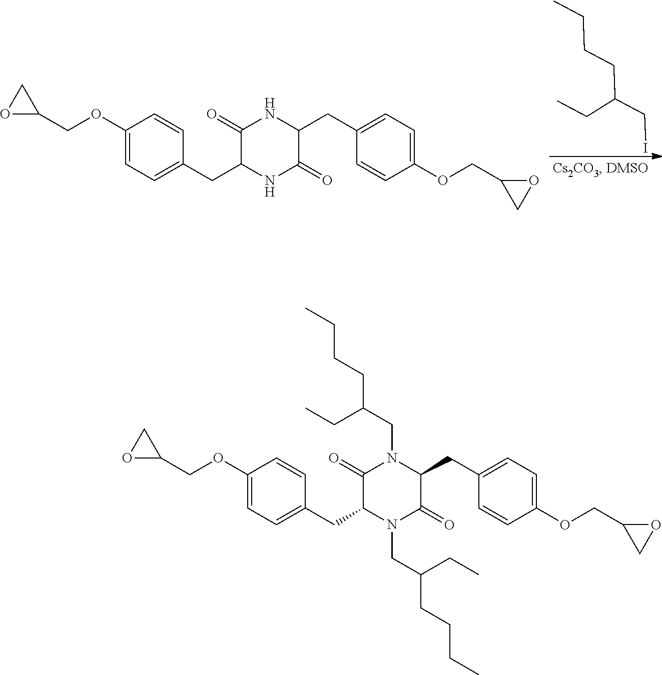

### Step 4: Preparation of 1,4-bis(2-ethylhexyl)-3,6-bis(4-(oxiran-2-ylmethoxy)benzyl)piperazine-2,5-dione

A 1 L single-neck reactor was charged with 3,6-bis(4-(oxiran-2-ylmethoxy)benzyl) piperazine-2,5-dione (10 g, 22.83 mmol) and dry DMSO (100 mL). The solution was stirred at ambient temperature for 30 minutes until a clear solution was obtained. Cesium carbonate (33.5 g, 102.7 mmol) was added, and the stirring was continued for 30 minutes. Then, 3-ethyl-1-iodohexane (14.4 mL, 79.9 mmol) was added to the mixture, and the reaction mixture was stirred for 2 days. After 2 days, the HPLC of the aliquot showed more than 90% of the starting material was converted. The reaction mixture was filtered to remove the solids, the solids were rinsed with MTBE (100 mL), and the filtrate was slowly poured into 120 ml of ice cold water. The organic layer was separated and washed with 80 ml of water and 80 ml of brine. The solution was dried over sodium sulfate and concentrated under vacuum to yield the crude product as a yellow oil, which was purified by column chromatography using EtOAc/hexane/Et3N mixture. A yellow, clear oil was obtained. (Yield: 2.6 g, 17%) NMR-GLC 20547 ¹H NMR (CDCl₃): 7.03 (d, J=8.4 Hz, 2H), 6.87 (d, J=8.3 Hz, 2H), 4.13 (t, J=9.2 Hz, 3H), 3.90 (dd, J=11.0, 5.5 Hz, 2H), 3.26-3.31 (m, 1H), 2.85 (t, J=4.5 Hz, 2H), 2.68-2.71 (m, 1H), 2.26-2.40 (m, 2H), 1.26, 0.98 (m, 9H), 0.84 (t, J=7.2 Hz, 3H), 0.78 (t, J=7.4 Hz, 2H), 0.71 (t, J=7.1 Hz, 2H).

### Example 6: General Reaction for N-Alkylation

The foregoing method was repeated with 2-ethylhexyl iodide replaced for iodohexane, iodooctane, iododecane, and iodododecane; and the corresponding N-alkyl derivatives were obtained in yields between 17 and 53%.

For alkylation yielding the N-oleyl derivative, an Appel reaction procedure was implemented to prepare oleyl iodide. A round-bottom flask with stir bar was rendered dry by heating to 140° C. Based on a 10 gram scale of oleyl alcohol, 1.1 equivalent (eq.) of PPh₃, 1.2 eq of iodine, and 1.1 eq of imidazole were weighted out and added to the round bottom flask which was then closed with a septa. Then, 70 mL of DCM was added, and the mixture was stirred vigorously. Ten grams of oleyl alcohol were added dropwise to the mixture. The mixture took on a yellow-orange color. The reaction was stirred for 2 days. After the reaction was confirmed to have reached completion by TLC, 20 mL of solid thiosulfate (10% w/v) was added. The organic layer was collected and washed twice with 20 ml of sodium thiosulfate, followed by washings with 30 ml of water and 30 ml of brine, dried over magnesium sulfate, then filtered over paper. The filtrate was concentrated in vacuo to form a white solid. The white solid was triturated with pentane, filtered over glass wool and concentrated in vacuo to form a yellow oil.

### Example 7: Non-Limiting Stepwise Synthesis of p-Hydroxyphenyl-Glycine Dimer

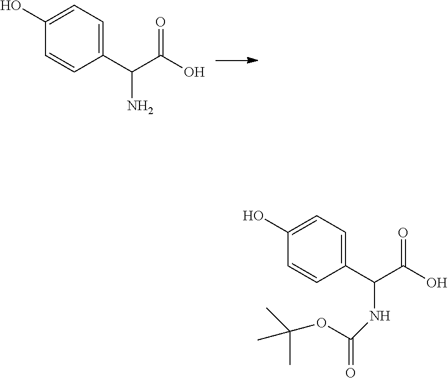 [SMILES: NC(C(=O)O)c1ccc(O)cc1 ; CC(C)(C)OC(=O)NC(C(=O)O)c1ccc(O)cc1]

(2R)-2-Amino-2-(4-hydroxyphenyl)acetic acid (1.00 eq, 1.00 g, 5.98 mmol) was dissolved in 1,4-dioxane (24 mL), water (24 ml), and 12.5 ml of an aqueous 2M NaOH solution in a 100 ml 2 neck flask under nitrogen. Di-tert-butyl dicarbonate (1.00 eq, 1.31 g, 5.98 mmol) was added to the solution dropwise, and the reaction was allowed to stir for 16 hours at room temperature. The reaction mixture was concentrated then acidified to pH 2 with 5M HCl, then extracted with ethyl acetate, and washed with a 5% sodium carbonate solution and brine. The organic layers were dried over magnesium sulfate, filtered, then concentrated in vacuo. Finally, 2-(tert-butoxycarbonylamino)-2-(4-hydroxyphenyl)acetic acid [4-HPG N-Boc] (1.08 g, 4.03 mmol, 67.36% yield) was isolated as a pink tacky solid and used in the next step without further purification.

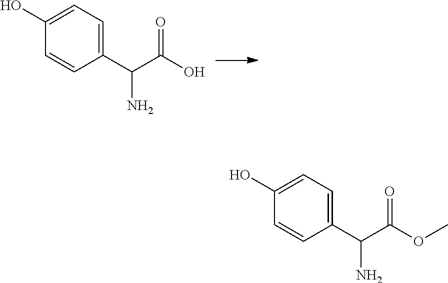

rac-(2R)-2-amino-2-(4-hydroxyphenyl)acetic acid (1.00 eq, 1.00 g, 5.98 mmol) was dissolved in 20 ml of 1.25M HCl in methanol and stirred at 70° C. for 3 hours. Then, the solvent was evaporated on a rotovap to yield 1.064 g of crude pink-white solid. This solid was washed with 250 ml of saturated sodium carbonate and extracted with ethyl acetate (4×100 ml) to provide 4-hydroxyphenyl-glycine methyl ester [4-HPG OMe]. (Yield: 33.766%, isolated 0.366 g of product).

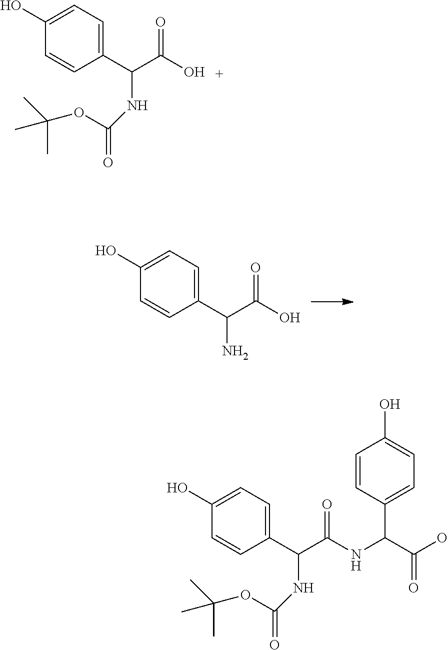 [SMILES: CC(C)(C)OC(=O)NC(C(=O)O)c1ccc(O)cc1 ; NC(C(=O)O)c1ccc(O)cc1 ; CC(C)(C)OC(=O)NC(C(=O)NC(C(=O)O)c1ccc(O)cc1)c1ccc(O)cc1]

4-hydroxyphenyl-glycine methyl ester (4-HPG OMe), 4-HPG N-Boc, HBTU, and DMAc were added to a 25 ml 2 neck round bottom flask and stirred for 15 min at room temperature under nitrogen. The reaction was cooled to 0° C., and trimethylamine (0.70 ml) was added dropwise over 15 min and then allowed to stir overnight. The reaction was then quenched with 2 ml of ice cold water, stirred for 10 mins and extracted 3× with EtoAc (2 ml). The organic layers were washed with 5% sodium carbonate and then brine, dried, and concentrated.

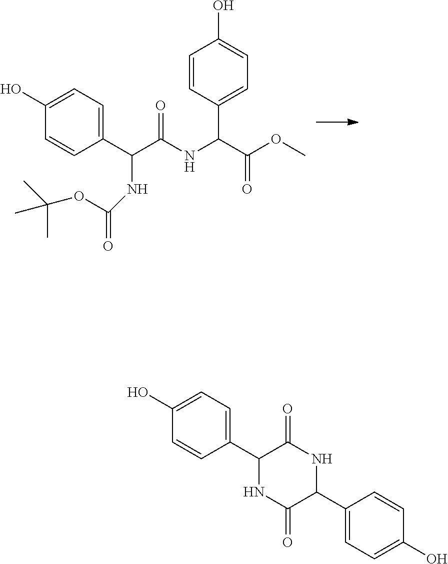 [SMILES: COC(=O)C(NC(=O)C(NC(=O)OC(C)(C)C)c1ccc(O)cc1)c1ccc(O)cc1 ; O=C1NC(c2ccc(O)cc2)C(=O)NC1c1ccc(O)cc1]

A 3 L single-neck reactor was charged with the foregoing dipeptide peptide (0.31 g) and formic acid (2.1 mL), and the mixture was stirred at ambient temperature for 5 h. Formic acid was removed under reduced pressure by azeotropic distillation with toluene. The residue was dissolved in sec-butanol (7.5 mL) and toluene (2.5 mL), and the solution was refluxed for 3 hours. The reaction mixture was concentrated to yield the crude material as a yellow-white solid.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

1. A composition comprising a structure having formula (I) or (II): 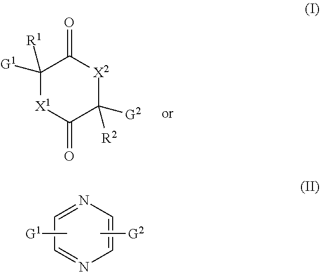
or a salt thereof, wherein:
each of G¹ and G² comprises, independently, hydroxyl, carboxyl, amino, amido, cyanato, isocyanato, cyano, isocyano, optionally substituted alkenyl, optionally substituted alkynyl, optionally substituted (hetero)cycloalkyl, or optionally substituted epoxy;
each of R¹ and R² is, independently, H or optionally substituted alkyl;
X¹ is oxy or —N—R^{g1}, wherein R^{g1} is H, optionally substituted alkyl, optionally substituted aryl, or optionally substituted aralkyl;
X² is oxy or —N—R^{g2}, wherein R^{g2} is H, optionally substituted alkyl, optionally substituted aryl, or optionally substituted aralkyl;
R^{g1} and G¹, taken together with the nitrogen to which R^{g1} is bound, can optionally form an optionally substituted heterocyclyl; and
R^{g2} and G², taken together with the nitrogen to which R^{g2} is bound, can optionally form an optionally substituted heterocyclyl.

2. The composition of claim 1, wherein:
G¹ is -L^{G1}-L^{G3}-R^{G1}, -L^{G1}-Ar^{G1}—R^{G1}, -L^{G1}-Het^{G1}-R^{G1}, or -L^{G1}-Ar^{G1}-L^{G3}-R^{G1};
G² is -L^{G2}-L^{G4}-R^{G2}, -L^{G2}-Ar^{G2}—R^{G2}, -L^{G2}-Het^{G2}-R^{G2}, or -L^{G2}-Ar^{G1}-L^{G4}-R^{G2};
each of L^{G1} and L^{G2} is, independently, a covalent bond, an amide bond, oxy, optionally substituted alkylene, optionally substituted alkenylene, optionally substituted heteroalkylene, optionally substituted arylene, optionally substituted (aryl)(alkyl)ene, optionally substituted heterocyclyldiyl, or optionally substituted (heterocyclyl)(alkyl)ene;
each of Ar^{G1} and Ar^{G2} is, independently, optionally substituted arylene or optionally substituted (aryl)(alkyl)ene;
each of Het^{G1} and Het^{G2} is, independently, optionally substituted heterocyclyldiyl or optionally substituted (heterocyclyl)(alkyl)ene;
each of L^{G3} and L^{G4} is, independently, a covalent bond, —NR^{N1}—, or oxy, wherein R^{N1} is H or optionally substituted alkyl; and
each of R^{G1} and R^{G2} is, independently, hydroxyl, carboxyl, amino, amido, cyanato, isocyanato, cyano, isocyano, optionally substituted alkenyl, optionally substituted alkynyl, optionally substituted (hetero)cycloalkyl, or optionally substituted epoxy.

3. The composition of claim 2, wherein the optionally substituted alkenyl has a structure of: 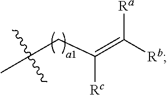
wherein each of R^{a}, R^{b}, and R^{c} is, independently, H, optionally substituted alkyl, or optionally substituted alkenyl; and wherein a1 is an integer of from 0 to 4.

4. The composition of claim 2, wherein the optionally substituted epoxy has a structure of: 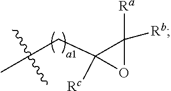
wherein each of R^{a}, R^{b}, and R^{c} is, independently, H, optionally substituted alkyl, or optionally substituted alkenyl; and wherein a1 is an integer of from 0 to 4.

5. The composition of claim 1, wherein the composition comprises a structure having formula (Ia): 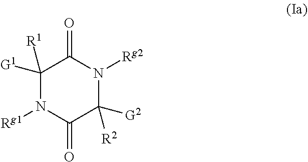
or a salt thereof, wherein:
each of R^{g1} and R^{g2} is, independently, H, optionally substituted alkyl, optionally substituted aryl, or optionally substituted aralkyl;
R^{g1} and G¹, taken together with the nitrogen to which R^{g1} is bound, can optionally form an optionally substituted heterocyclyl; and
R^{g2} and G², taken together with the nitrogen to which R^{g2} is bound, can optionally form an optionally substituted heterocyclyl.

6. The composition of claim 1, wherein the composition comprises a structure having formula (Ib): 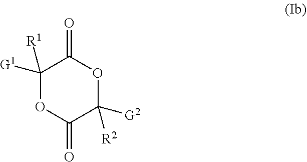
or a salt thereof.

7. The composition of claim 1, wherein the composition comprises a structure having formula (Ic): 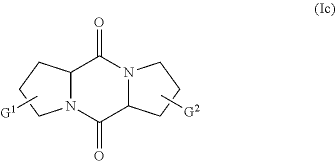
or a salt thereof.

8. The composition of claim 1, wherein the composition comprises a structure having formula (Id): 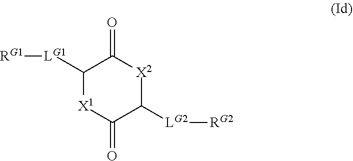
or a salt thereof, wherein:
each of L^{G1} and L^{G2} is, independently, a covalent bond, an amide bond, —NR^{N1}—, oxy, optionally substituted alkylene, optionally substituted alkenylene, optionally substituted heteroalkylene, optionally substituted arylene, optionally substituted heterocyclyldiyl, or optionally substituted (heterocyclyl)(alkyl)ene; and
each of R^{G1} and R^{G2} is, independently, hydroxyl, optionally substituted hydroxyalkyl, optionally substituted hydroxyaryl, carboxyl, amino, optionally substituted aminoalkyl, optionally substituted aminoaryl, amido, cyanato, isocyanato, cyano, isocyano, optionally substituted alkenyl, optionally substituted alkynyl, optionally substituted (hetero)cycloalkyl, or optionally substituted epoxy.

9. The composition of claim 1, wherein the composition comprises a structure selected from the group consisting of: 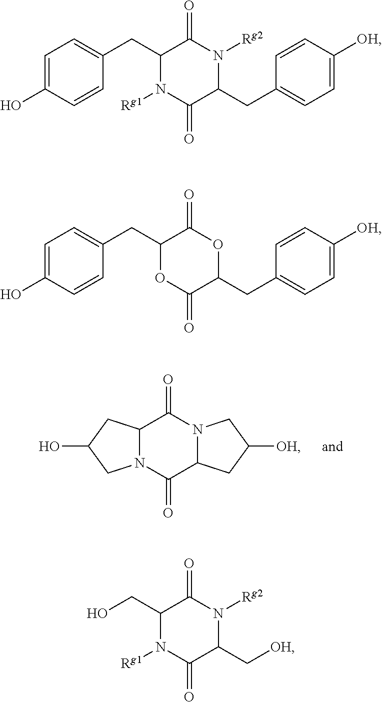
or a salt thereof.

10. The composition of claim 1, wherein the composition comprises a structure selected from the group consisting of: 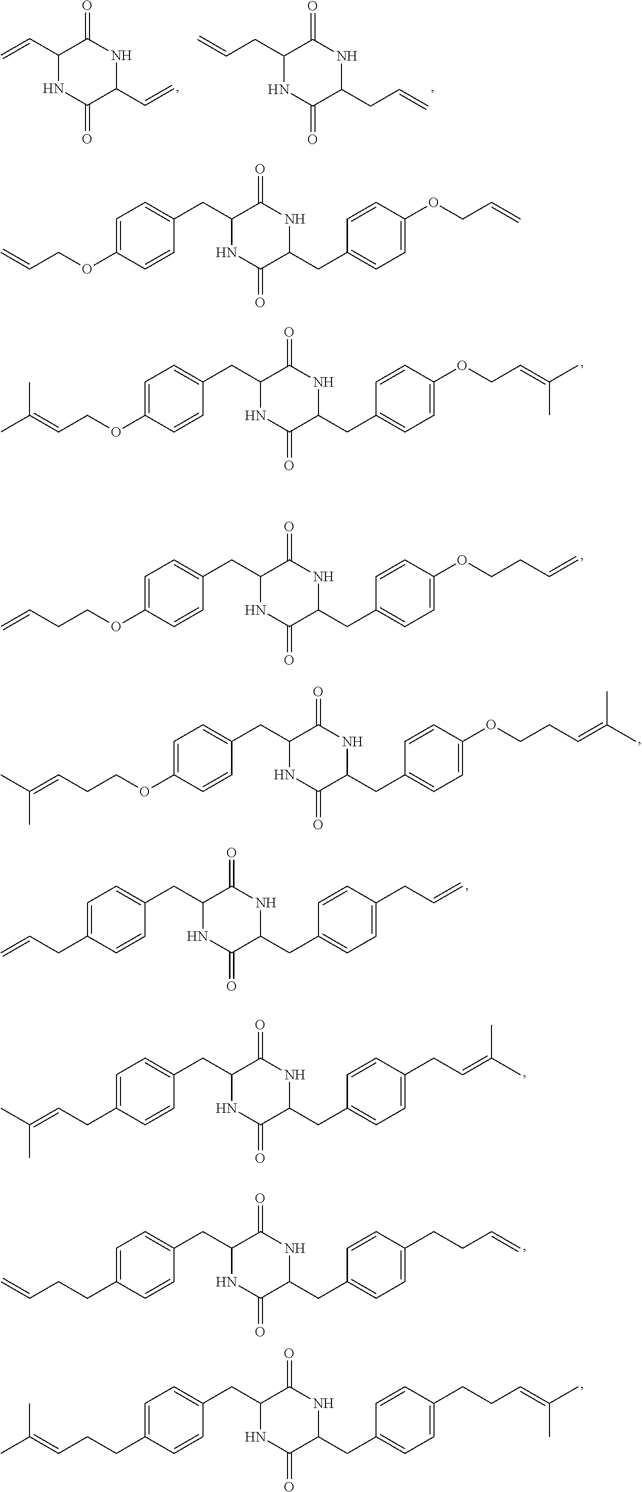 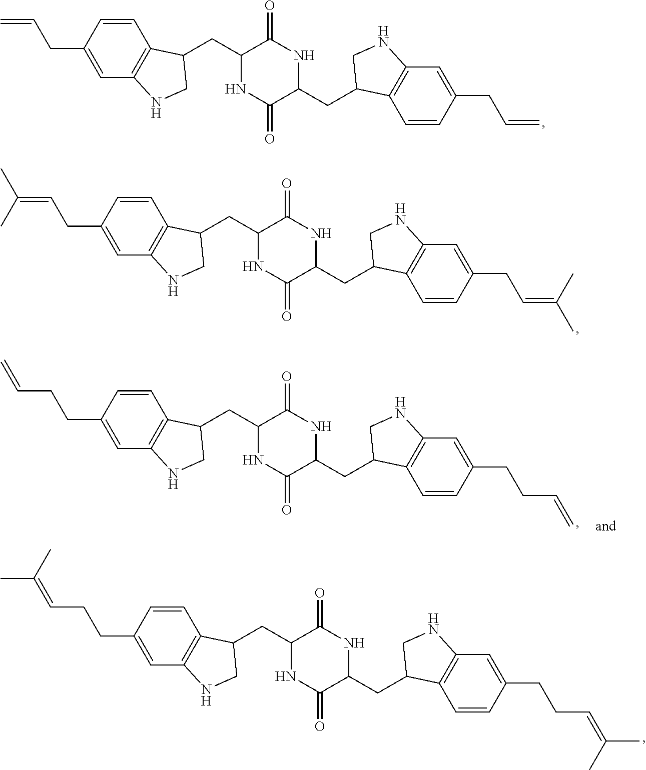
or a salt thereof.

11. The composition of claim 1, wherein the composition comprises a structure selected from the group consisting of: 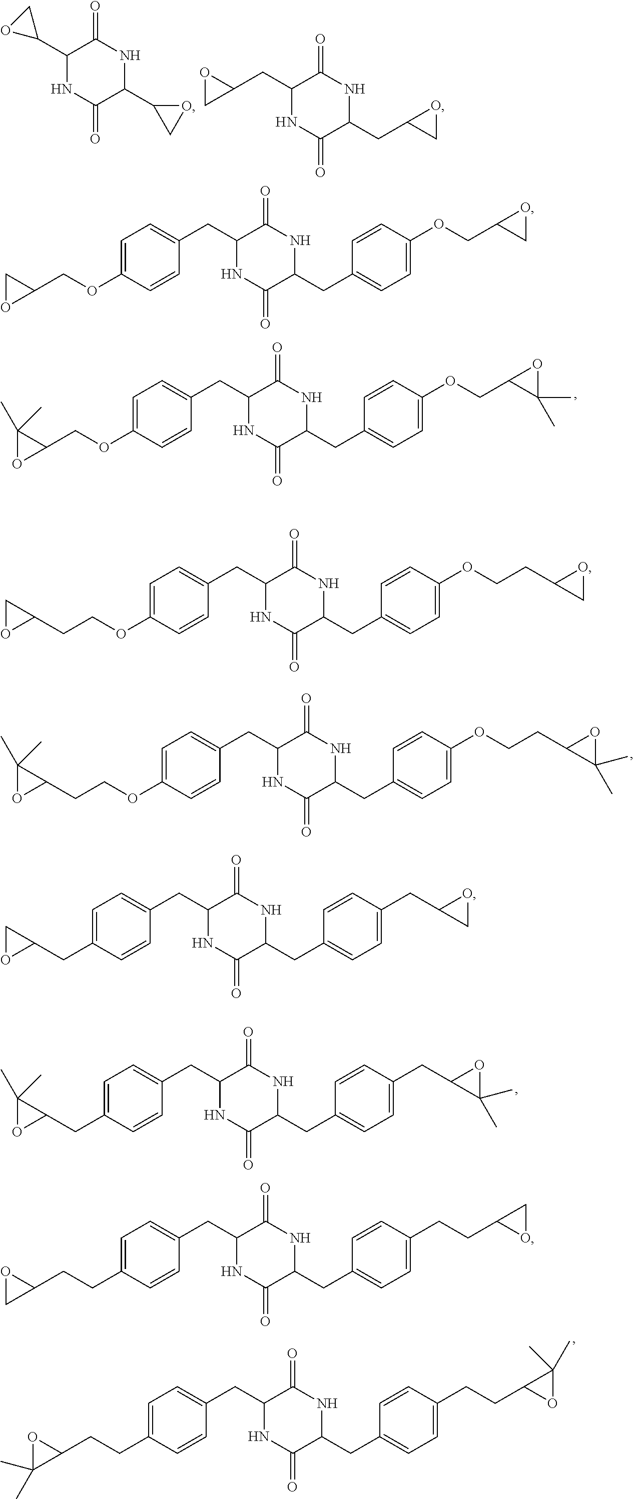 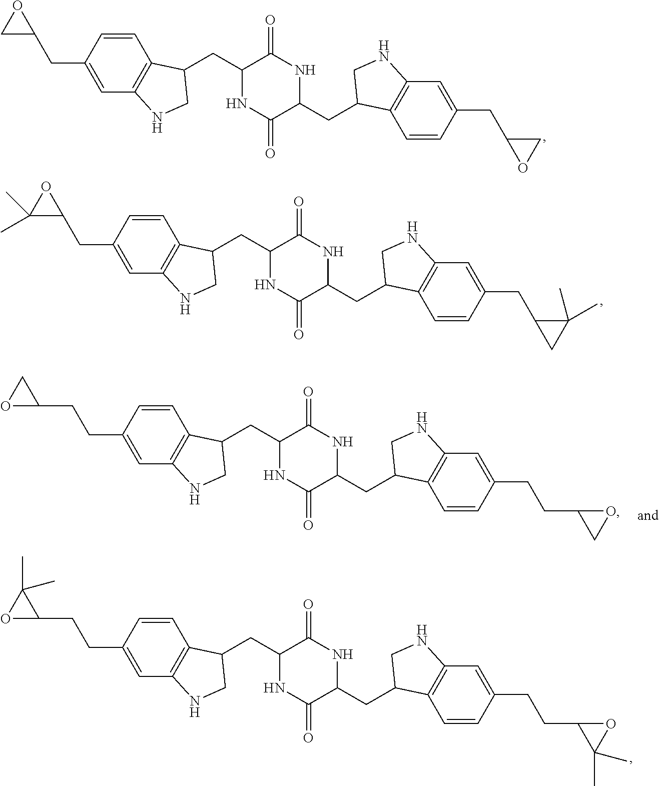
or a salt thereof.

12. The composition of claim 1, wherein the composition is a monomer, a polymer, or a copolymer.

13. A method of making a composition of claim 1, the method comprising:
providing a first amino acid and a second amino acid, wherein the first and second amino acids are selected from the group consisting of hydroxymandelic acid, hydroxyproline, serine, and tyrosine; and
forming a dimer between the first and second amino acids.

14. The method of claim 13, thereby producing a monomer for a copolymer.

15. A method of making a composition of claim 1, the method comprising:
providing a first amino acid and a second amino acid, wherein the first and second amino acids are selected from the group consisting of tyrosine, tryptophan, phenylalanine, vinylglycine, allylglycine, and a derivative thereof comprising an optionally substituted alkenyl; and
forming a dimer between the first and second amino acids; and
optionally epoxidizing the dimer in the presence of an oxidant.

16. The method of claim 15, wherein the first and second amino acids are selected from the group consisting of L-vinylglycine, L-allylglycine, O-allyl-L-tyrosine, O-buten-3-enyl-L-tryrosine, O-(3-methyl-but-2-enyl)-L-tryrosine, O-(4-methyl-pent-3-enyl)-L-tryrosine, 4-allyl-L-phenylalanine, 4-but-3-enyl-L-phenylalanine, 6-allyl-L-tryptophan, and 6-(3-methylbut-2-enyl)-L-tryptophan, or a salt thereof.

17. The method of claim 15, thereby producing an ion-free epoxy resin.

18. The method of claim 17, wherein the total ion content is less than 1 part per thousand.

19. A method of making a composition of claim 1, the method comprising:
providing an organism a plurality of amino acids, thereby producing a plurality of prenylated amino acids; and
forming a dimer between two of the plurality of amino acids.

20. A film comprising a composition of claim 1.

21. The film of claim 20, wherein the film is an adhesive or a coating.

22. A composite or bulk structure comprising a composition of claim 1.

23. A fiber or a particle comprising a composition of claim 1.
